(19) United States
(12) Patent Application Publication
(10) Pub. No.: US 2023/0416692 A1
(43) Pub. Date: Dec. 28, 2023

(54) REVERSE GENETIC SYSTEM FOR SARS-COV-2

(71) Applicant: BOARD OF REGENTS, THE UNIVERSITY OF TEXAS SYSTEM, Austin, TX (US)

(72) Inventors: PEI-YONG SHI, Galveston, TX (US); VINEET MENACHARY, Galveston, TX (US); Xuping Xie, Galveston, TX (US)

(21) Appl. No.: 17/914,735

(22) PCT Filed: Mar. 26, 2021
(86) PCT No.: PCT/US2021/024532

§ 371 Date: Sep. 26, 2022

Related U.S. Application Data
(60) Provisional application No. 63/000,713, filed on Mar. 27, 2020.
(60) Provisional application No. 63/041,667, filed on Jun. 19, 2020.

(51) Int. Cl. C12N 7/00; C12Q 1/18; C07K 14/46; C12N 9/02

(52) U.S. Cl. CPC C12N 7/00; C12Q 1/18; C07K 14/46; C12N 9/0069; C12Y 113/12013; C12N 2770/20021

(57) ABSTRACT

Certain embodiments of the invention include recombinant reverse genetic systems for SARS-COV-2 virus.

### PRIORITY

This application claims priority to U.S. Provisional Application Ser. No. 63/000,713 filed Mar. 27, 2020 and U.S. Provisional Application Ser. No. 63/041,667 filed Jun. 19, 2020, each of which is incorporated herein by reference in its entirety.

### US FEDERAL GOVERNMENT FUNDING

This invention was made with government support under AI134907 awarded by the National Institutes of Health. The government has certain rights in the invention.

### BACKGROUND

Severe acute respiratory syndrome coronavirus 2 (SARS-CoV-2) emerged in early 2020 with human cases in Wuhan, China (Gralinski and Menachery, 2020 Viruses 12(2):135). It has rapidly rampaged worldwide, causing a pandemic of coronavirus disease (COVID-19) that ranges from fever and breathing difficulty to acute respiratory distress and death (Huang et al., 2020; Zhu et al., 2020). With over 300,000 people infected in less than 3 months, SARS-CoV-2 causes the most severe disease in older patients and people with comorbidities, including heart disease, diabetes, and other health conditions (Wu and McGoogan, 2020, JAMA 323(13):1239-42). These findings correspond closely to the 2003 coronavirus severe acute respiratory syndrome (SARS-CoV) and Middle East respiratory syndrome (MERS-CoV), which has emerged since 2012 (Assiri et al., 2013; Huang et al., 2020). On Jan. 30, 2020, the International Health Regulations Emergency Committee of the World Health Organization declared the outbreak a public health emergency of international concern (PHEIC). On Jan. 31, 2020 a public health emergency (PHE) was declared for the United States. Importantly, with massive hospitalization rates and high mortality, SARS-CoV-2 remains a major threat to humankind and intervention strategies are being rapidly pursued.

A key tool in responding to novel emergent viruses is the generation of reverse genetic systems to explore and characterize new pathogens. Classically, reverse genetic systems for coronaviruses have been complicated by their large genome size (30,000 nucleotides) and the existence of bacteriotoxic elements in their genome that make them difficult to propagate (Almazan et al., 2014, Virus Research 189:262-70). Several approaches have been devised to overcome this barrier, such as multiple plasmid systems to disrupt toxic elements and to reduce deletions/truncations (Yount et al., 2002, J Viral 76(21):11065-78). Using this approach, researchers have developed infectious clones for several coronaviruses, including SARS-CoV, MERS-CoV, and others (Menachery et al., 2015, Nat Med 21(12):1508-13; Menachery et al., 2016, PNAS 113(11):3048-53; Scobey et al., 2013a, PNAS 110(40):16157-62; Yount et al., 2003). Thao et al. recently reported a yeast-based synthetic genomics platform for rapid construction of infectious clones for murine hepatitis coronavirus (MHV-CoV), MERS-CoV, and SARS-CoV-2 (Thao et al., 2020). However, the yeast platform-produced SARS-CoV-2 has not been fully characterized for its biological properties (e.g., replication kinetics) in comparison with its original clinical isolate. Such characterization is essential for ensuring the quality of the genetic system to rescue recombinant viruses that recapitulate the biological features of their corresponding clinical isolates. Once validated, the reverse genetic systems allow rapid characterization of novel viruses, development of reporter viruses, and generation of live-attenuated vaccine candidates to respond to emerging infections. Together with animal pathogenesis models, reverse genetic systems offer powerful tools needed to characterize, understand, and respond to emerging virus outbreaks.

There is a need for improved methods of identifying the presence of the SARS-CoV-2 virus.

### SUMMARY

The COVID-19 pandemic caused by severe acute respiratory syndrome coronavirus 2 (SARS-CoV-2) underscores the urgency to develop experimental systems for studying this virus and variants thereof, and to develop countermeasures. In response to the ongoing pandemic of SARS-CoV-2, the inventors have developed a robust reverse genetic system for SARS-CoV-2 and a reporter virus (for example mNeonGreen (e.g., SEQ ID NO:4 and SEQ ID NO:5) or nanoluciferase reporter virus (e.g., SEQ ID NO:6 and SEQ ID NO:7)). Recombinant virus derived from the system recapitulates the replication kinetics of the original clinical isolates. In addition, the reporter remains stable for at least five passages, allowing its use in long-term studies. Using type-I interferon, the inventors demonstrated that the reporter virus could be reliably used to study viral replication and pathogenesis as well as to develop vaccine and antiviral drugs.

Herein a reverse genetic system for SARS-CoV-2 is described. Seven cDNA fragments spanning the SARS-CoV-2 genome were assembled to a full-genome cDNA. RNA transcribed from the full-genome cDNA was highly infectious after electroporated into cells, producing 2.9×10⁶ PFU/ml of virus. Compared with the clinical isolate, the infectious clone-derived SARS-CoV-2 (icSARS-CoV-2) exhibited similar plaque morphology, viral RNA profile, and replication kinetics. The icSARS-CoV-2 retained engineered molecular markers without other mutations. The rSARS-CoV-2 can be a reporter SARS-CoV-2. The reporter SARS-CoV-2 expresses a reporter molecule or protein when infecting a cell. In certain aspects the reporter protein is mNeonGreen (e.g., SEQ ID NO:4/5) protein (icSARS-CoV-2-mNG) or nanoluciferase (e.g., SEQ ID NO:6/7) (e.g., NanoLuc™ Promega Corp.) (SARS-CoV-2-Nluc) or firefly luciferase (e.g., SEQ ID NO:8/9).

A stable mNeonGreen SARS-CoV-2 (icSARS-CoV-2-mNG) or nanoluciferase SARS-CoV-2 (SARS-CoV-2-Nluc) was produced by engineering the reporter gene into the OFR7 of the viral genome. An icSARS-CoV-2-mNG or SARS-CoV-2-Nluc can be used to evaluate the antiviral activities of interferon and to screen inhibitors. Collectively, the reverse genetic system and reporter virus have provided key reagents to study SARS-CoV-2 and develop countermeasures.

Embodiments of the invention are directed to stable full-length cDNA clones of SARS-CoV-2. The cDNA clone-derived SARS-CoV-2 described herein was virulent. Furthermore, the recombinant virus was highly infectious. These experimental systems are essential to study viral pathogenesis and vector transmission as well as to develop a SARS-CoV-2 vaccine, etc.

In certain aspects, the reverse genetic system described, e.g., icSARS-CoV-2-mNG, or SARS-CoV-2-Nluc, and other reporter constructs (e.g., luciferase or fluorescent genes), can be used to produce recombinant SARS-CoV-2 viruses. In certain aspects, recombinant viruses and/or recombinant virus production can be used to determine animal or human neutralizing antibody titers for serodiagnosis, vaccine evaluation, and antiviral drug discovery.

In other aspects, the reverse genetic system described can be used to develop/engineer live-attenuated vaccine(s) for prevention or amelioration of infection by SARS-CoV-2 and other related coronaviruses. The attenuation can be derived from mutations and/or deletions of viral genome as well as insertions of non-viral sequences.

In certain aspects, replicons can be produced/engineered by deletion of portions of the viral genome and the addition of heterologous nucleic acids such as reporter genes (e.g., luciferase gene, green fluorescent protein, and other fluorescent genes) as well as antibiotic resistance gene such as Neo. These replicons and their replicon-containing cell lines can be used for antiviral drug discovery.

In other aspects, a replicon can be used to develop or engineer virus-like particles (VLPs) through trans complementation of the deleted regions/genes in cells. The VLPs can be used for vaccine candidates for SARS-CoV-2. The VLPs can also be used for antiviral drug discovery.

Certain embodiments are directed to a reverse genetic system of SARS-CoV-2. In certain aspects the SARS-COV-2 nucleic acids can have at least 90, 95, 98, 99, 99.99, or 100% sequence identity to SEQ ID NO:1, SEQ ID NO:2, or SEQ ID NO:3 or any 10, 20, 30, 40, 50, 100, 200, 300, 400, 500, 600, 700, 800, 1000, 2000, 3000, 4000, 5000, 6000, 7000, 8000, 9000, 10000, 11000 to 29900 consecutive nucleotide segment thereof, including all values and ranges there between. In certain aspects, a SARS-CoV-2 nucleic acid sequence has a sequence that is at least 98% identical to SEQ ID NO:1, SEQ ID NO:2, or SEQ ID NO:3. In certain aspects, a SARS-CoV-2 nucleic acid sequence has a sequence that is 100% identical to SEQ ID NO:1, SEQ ID NO:2, or SEQ ID NO:3. In certain aspects and as an example, a nucleotide segment encoding a reporter protein can be inserted in place of nucleotides 27,394 to 27,759 of SEQ ID NO:1 or a corresponding segment in another coronavirus vector.

The SARS-CoV-2 nucleic acids can be isolated or recombinant nucleic acids (e.g., DNA) or included in a recombinant SARS-CoV-2 replicon, a virus, a SARS-CoV-2, a viral particle, a SARS-CoV-2 particle, an expression cassette, a host cell, a SARS-CoV-2 vector, and the like. In still a further aspect, a SARS-CoV-2 nucleic acid sequence can comprise a heterologous nucleic acid segment. In certain aspects, the heterologous nucleic acid segment can encode a marker (e.g., a reporter protein). In certain aspects the reporter protein is a fluorescent protein, such as a green fluorescent protein (mNeonGreen) or nanoluciferase (Nluc).

Embodiments are directed to SARS-CoV-2 comprising all or part of the SARS-COV-2 nucleic acid sequence of SEQ ID NO: 1, SEQ ID NO:2, or SEQ ID NO:3. In certain aspects the SARS-CoV-2 is a recombinant SARS-CoV-2. Certain embodiments are directed to a SARS-CoV-2 having a genome comprising (a) a SARS-CoV-2 nucleic acid segment that is at least 95, 98, 99, or 100% identical to SEQ ID NO:1, SEQ ID NO:2, or SEQ ID NO:3 and (b) a heterologous nucleic acid segment. In certain aspects the heterologous segment is inserted in place of nucleotides 27,394 to 27,759 of SEQ ID NO:1 or a corresponding segment in another coronavirus vector

The term “coronavirus” refers to a virus whose genome is plus-stranded RNA of about 27 kb to about 33 kb in length depending on the particular virus. The virion RNA has a cap at the 5′ end and a poly A tail at the 3′ end. The length of the RNA makes coronaviruses the largest of the RNA virus genomes. Coronavirus RNAs can encode: (1) an RNA-dependent RNA polymerase; (2) N-protein; (3) three envelope glycoproteins; and (4) three non-structural proteins. These coronaviruses infect a variety of mammals and birds. They cause respiratory infections (common), enteric infections (mostly in infants >12 mo.), and possibly neurological syndromes. Coronaviruses are transmitted by aerosols of respiratory secretions. Coronaviruses are exemplified by, but not limited to, human enteric SARS-CoV-2 (GenBank accession number NC 045512.2), coV (ATCC accession #VR-1475), human coV 229E (ATCC accession #VR-740), human coV OC43 (ATCC accession #VR-920), and SARS-coronavirus (Center for Disease Control).

The term “nucleic acid” refers to a polymeric compound comprising nucleosides or nucleoside analogs which have nitrogenous heterocyclic bases or base analogs linked together to form a polynucleotide, including conventional RNA, DNA, mixed RNA-DNA, and polymers that are analogs thereof. A nucleic acid “backbone” may be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid may be ribose, deoxyribose. Nitrogenous bases may be conventional bases (A, G, C, T, U), analogs thereof (e.g., inosine or others; see The Biochemistry of the Nucleic Acids 5-36, Adams et al., ed., 11th ed., 1992)

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins.

The term “recombinant” refers to an artificial combination of two otherwise separated segments of nucleic acid, e.g., by chemical synthesis or by the manipulation of isolated segments of nucleic acids by genetic engineering techniques.

The term “SARS-CoV-2 replicon” is used to refer to a nucleic acid molecule expressing SARS-CoV-2 genes such that it can direct its own replication (amplification).

The term “SARS-CoV-2 replicon particle” refers to a virion or virion-like structural complex incorporating a SARS-CoV-2 replicon.

The term “SARS-CoV-2 reporter virus” refers to a virus that is capable of directing the expression of a sequence(s) or gene(s) of interest. The reporter construct can include a 5′ sequence capable of initiating transcription of a nucleic acid encoding a reporter molecule or protein such as luciferase, fluorescent protein, Neo, SV2 Neo, hygromycin, phleomycin, histidinol, and DHFR. The reporter virus can be used an indicator of infection of a cell by a SARS-CoV-2 virus.

The term “expression vector” refers to a nucleic acid that is capable of directing the expression of a sequence(s) or gene(s) of interest. The vector construct can include a 5′ sequence capable of initiating transcription of a nucleic acid, e.g., all or part of a SARS-CoV-2 virus. The vector may also include nucleic acid molecule(s) to allow for production of virus, a 5′ promoter that is capable of initiating the synthesis of viral RNA in vitro from cDNA, as well as one or more restriction sites, and a polyadenylation sequence. In addition, the constructs may contain selectable markers such as Neo, SV2 Neo, hygromycin, phleomycin, histidinol, and DHFR. Furthermore, the constructs can include plasmid sequences for replication in host cells and other functionalities known in the art. In certain aspects the vector construct is a DNA construct.

The term “expression cassette” refers to a nucleic acid segment capable of directing the expression of one or more nucleic acids, or one or more nucleic acids that are in turn translated into an expressed protein.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve the methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

FIG. 1A-1F. Assembly of a full-length SARS-CoV-2 infection cDNA clone. (A) Genome structure SARS-CoV-2. The open reading frames (ORFs) from the full genome are indicated. (B) Strategy for in vitro assembly of an infectious cDNA clone of SARS-CoV-2. The nucleotide sequences and genome locations of the cohesive overhangs are indicated. The wild-type full-length cDNA of SARS-CoV-2 (IC WT) was directionally assembled using in vitro ligation. (C) Diagram of the terminal sequences of each cDNA fragment recognized by BsaI and Esp3I. (D) Gel analysis of the seven purified cDNA fragments. Individual fragments (F1 to F7) were digested from corresponding plasmid clones and gel-purified. Seven purified cDNA fragments (50-100 ng) were analyzed on a 0.6% native agarose gel. The 1-kilobase (kb) DNA ladders are indicated. (E) Gel analysis of cDNA ligation products. About 400 ng of purified ligation product was analyzed on a 0.6% native agarose gel. Triangle indicates the full-length (FL) cDNA product. Circles indicate the intermediate cDNA products. (F) Gel analysis of RNA transcripts. About 1 μg of in vitro transcribed (IVT) RNAs were analyzed on a 0.6% native agarose gel. Triangle indicates the genome-length RNA transcript. Circles show the shorter RNA transcripts.

FIG. 2A-2F. Characterization of the wild-type icSARV-CoV-2 (IC WT). (A) Bright-field images of the Vero E6 cells electroporated with RNA transcripts. Cytopathic effects (CPE) appeared in the IC WT RNA-transfected cells on day 4 post-transfection. The titer of the P0 virus (directly from the transfected cells) is shown in plaque-forming units (PFU) per ml. (B) Plaque morphology of the original clinical isolate (WA1=2019-nCoV/USA_WA1/2020) and the recombinant P1 IC WT virus. Plaques were developed in Vero E6 cells on day 2 post-infection. (C) Replication kinetics. Vero E6 cells were infected with the clinical isolate or recombinant P1 IC WT virus at MOI 0.01. Viruses in culture fluids were quantified by plaque assay. Results from triplicate experiments were presented with error bars indicating standard deviations. (D) Northern blot analysis of full-length and subgenomic RNAs. Numbers indicated the FL (band 1) and eight subgenomic RNAs (bands 2-9). (E) Sequence differences between the original clinical isolate WA1 and the recombinant P1 IC WT. The three silent nucleotide changes were engineered as molecular markers. (F) Chromatograms of Sanger sequencing results. The engineered molecular maker mutations are indicated.

FIG. 3A-3F. Generation of a mNeonGreen SARS-CoV-2. (A) Assembly of the full-length mNeonGreen (mNG) SARS-CoV-2 cDNA. The mNG gene was placed downstream of the regulatory sequence of ORF7a to replace the ORF7a sequence (Sims et al., 2005) in the subclone F7. (B) Plaque morphology of the P1 IC mNG virus. Plaques were developed in Vero E6 cells on day 2 post-infection. (C) Replication kinetics. Vero E6 cells were infected with the wild-type icSARS-CoV-2 (IC WT) or reporter icSARS-CoV-2-mNG (IC mNG) at MOI of 0.01. Viruses in culture medium were quantified by plaque assay. (D) Fluorescence microscopy analysis of P1 mNG virus-infected cells. Vero E6 cells were infected with P1 mNG viruses at MOI of 0.3. Representative mNeonGreen-positive (green) images are shown. (E) Kinetics of fluorescence intensity. Vero E6 cells were infected with MOI of 1.0, 0.3 or 0.1. After background signal subtraction, the fluorescence intensities from 12 to 48 h post-infection are shown. Results from triplicate experiments were presented with bars representing standard deviations. (F) Summary of full-genome sequence of mNG virus (P1 IC mNG). Nucleotides different from the original clinical isolate (WA1) are indicated.

FIG. 4A-4E. Stability and application of mNeonGreen virus. The stability of mNG virus was analyzed by comparing the fluorescent signals between the cells infected with P1 and P5 reporter viruses. The presence of mNG gene in the P1 and P5 reporter viruses was also verified using RT-PCR. The application of mNG virus was examined by testing the antiviral activity of IFN-α treatment. (A) Fluorescence microscopy analysis of the P1 and P5 mNG virus-infected cells. (B) Gel analysis of mNG virus stability. Top panel depicts the theoretical results of RT-PCR followed by restriction enzyme digestion. Bottom panel shows the gel analysis of the RT-PCR products before (lanes 1-3) and after BsrGI/StuI digestion (lanes 4-6). About 100 ng DNA samples were analyzed on a 0.6% agarose gel. The DNA sizes are indicated. (C) Schematic diagram of IFN-α treatment. (D) Representative fluorescence images of reporter virus-infected cells after IFN-α treatment. The doses of IFN-α treatment are indicated. (E) Dose response curve of mNG signal inhibited by IFN-α. The Hillslope and EC₅₀ values are indicated. Results from triplicate experiments were presented with bars representing standard deviations.

FIG. 5A-5H. Development and characterization of SARS-CoV-2-Nluc. (A) Assembly of the full-length SARS-CoV-2-Nluc cDNA. The Nanoluciferase (Nluc) gene together with a PacI site was placed downstream of the regulatory sequence of ORF7 to replace the ORF7 sequence. (B) Plaque morphologies of infectious clone derived P1 SARS-CoV-2-Nluc (P1 IC Nluc) and wild-type SARS-CoV-2 (IC WT). (C) Replication kinetics. Vero E6 cells were infected with infectious clone derived IC WT or P1 IC Nluc at MOI 0.01. Viruses in culture supernatants were quantified by plaque assay. (D) Plaque morphology of P5 IC Nluc. (E) Replication kinetics of P5 IC Nluc on Vero E6 cells. (F) Luciferase signals produced from SARS-CoV-2-Nluc-infected Vero E6 cells at 12 h post-infection. Cells were infected with viruses at MOI 0.1. (G) Gel analysis of IC Nluc virus stability. The left panel depicts the theoretical results of RT-PCR followed by restriction enzyme digestion. The right panel shows the gel analysis of the RT-PCR products before (lanes 1-3) and after BsrGI/PacI digestion (lanes 4-6). (H) Summary of full-genome sequences of P1 and P5 IC Nluc viruses. Nucleotide and amino acid differences from the IC WT are indicated.

FIG. 6A-6E. Application of SARS-CoV-2-Nluc in analyzing hACE2 as an entry receptor. (A) Replication kinetics of SARS-CoV-2-Nluc (IC Nluc) on Vero E6 cells. Cells were infected with IC Nluc at MOI 1.0. At given time points, cells were harvested for luciferase signal measurement. The means and standard deviations from three independent experiments are presented. (B) Diagram to analyze hACE2 for IC Nuc entry. (C) Relative luciferase signals following infection of cells that were preincubated with anti-hDPP4 or anti-hACE2 antibodies. The luciferase signals from antibody-treated groups were normalized to those from untreated groups. The average and standard deviation of three independent experiments are presented. (D) Immunofluorescence analysis of hACE2 expression in A549-hACE2 cells. At 24 h post-seeding, the cells were fixed and stained with anti-hACE2 polyclonal antibody. (E) Luciferase signals from IC Nluc infected-A549 and A549-hACE2 cells. Cells were infected with indicated MOIs and luciferase signals were measured at 24 h post-infection.

FIG. 7A-7D. A rapid SARS-CoV-2-Nluc-based neutralization assay. (A) Schematic of the rapid neutralization assay. (B) Summary of neutralizing titers as measured by PRNT and SARS-CoV-2-Nluc neutralization (Nluc-NT) assay. Serum specimens 1-21 were from COVID-19 patients with confirmed prior RT-PCR diagnosis. Serum specimens 22-30 were from non-COVID-19 individuals. (C) Representative neutralizing curves of the Nluc-NT assay. The means and standard deviations from two independent experiments are shown. (D) Correlation analysis between the Nluc-NT₅₀ and PRNT₅₀ values. The correlation efficiency R 2 and p value calculated from a linear regression analysis are shown.

FIG. 8A-8E. SARS-CoV-2-Nluc-based antiviral screening. (A) Cytotoxicity of chloroquine on Vero E6 and A549-hACE2 cells. (B) Cytotoxicity of remdesivir on Vero E6 and A549-hACE2 cells. (C) Potency of chloroquine against SARS-CoV-2-Nluc on Vero E6 and A549-hACE2 cells. (D) Potency of remdesivir against SARS-CoV-2-Nluc on Vero E6 and A549-hACE2 cells. (E) Summary of CC₅₀, EC₅₀, and selectivity index (SI).

FIG. 9A-9E. A high-throughput neutralizing antibody assay for COVID-19 diagnosis. (A) Diagram of the cDNA constructs of wild-type (WT) SARS-CoV-2 (top panel) and mNG SARS-CoV-2 (bottom panel). The nucleotide positions of viral genome where mNG is engineered are indicated. (B) Assay flowchart. mNG SARS-CoV-2 was neutralized with COVID-19 patient sera. Vero CCL-81 cells were infected with the reporter virus/serum mixture with an MOI of 0.5. The fluorescence of infected cells was quantified to estimate the NT₅₀ value for each serum. (C) Representative images of reporter virus-infected Vero CCL-81 cells. Images for a positive neutralizing serum (top panel) and no serum control (bottom panel) are presented. The means from two independent experiments are presented. (D) Neutralization curves. Representative neutralization curves are presented for three positive sera and one negative sera. (E) Correlation analysis of NT₅₀ values between the reporter virus and PRNT assays. The Pearson correlation efficiency R² and p value (two-tailed) are indicated.

FIG. 10A-10K. Analysis of neutralizing activities of human sera using mNG SARS-CoV-2. (A-J) Neutralization curves for 60 specimens from patients confirmed with RT-PCT test positive. (K) Relative infection rate of mNG SARS-CoV-2 for 60 COVID-19-positive and 60 COVID-19-negative human sera at dilution of 20 folds.

### DESCRIPTION

The SARS-CoV-2 virus is a betacoronavirus, similar to MERS-CoV and SARS-CoV. All three of these viruses have their origins in bats. The sequences of viruses isolated from U.S. patients are similar to the virus sequences initially posted by China.

Described herein is the development of a full-length infectious clone of SARS-CoV-2 using a contiguous panel of cDNAs, class IIS restriction endonuclease, in vitro ligation, and in vitro transcription to generate full-length viral genome equivalent to the sequence of a clinical isolate. Next, full-length RNA was electroporated to produce recombinant SARS-CoV-2 with replication properties equivalent to the original clinical isolate. A similar approach was used to generate a reporter virus expressing mNG in place of SARS-CoV-2 ORF7. This reporter virus maintained indistinguishable replication properties to the wild-type icSARS-CoV and stably maintained the reporter gene through five passages. Finally, the utility of the reporter virus is described for evaluating the therapeutic efficacy of a potential therapy, e.g., type-I IFN, against SARS-CoV-2 infection. Overall, a key reagent to study SARS-CoV-2 is described herein. The reporter virus system will enable a myriad of approaches to high-throughput antiviral discovery.

One utility of the described reverse genetic system is to facilitate antiviral testing and therapeutic development. The icSARS-CoV-2 mNG reporter virus allows the use of fluorescence as a surrogate readout for viral replication. Compared with a standard plaque assay or TCID₅₀ quantification, the fluorescent readout shortens the assay turnaround time by several days. In addition, the fluorescent readout offers a quantitative measure that is less labor-intensive than the traditional means of viral titer reduction. Furthermore, the mNG virus-based assay could be automated in a high-throughput format to screen and test compounds directly against viral replication. As a proof-of-concept, it was demonstrated that, after treatments with type-I IFN, the reporter virus reliably revealed efficacy in a rapid and efficient manner. A similar study using plaque assay or TCID₅₀ would require significantly more time and labor to complete. In addition, the stability of the mNG reporter virus allows it to be used for longer-term studies and in vivo without fear of losing its fluorescent marker. Thus, this reporter virus offers a huge advantage for research community and pharmaceutical companies to develop therapeutics for COVID-19.

The reverse genetic system described herein represents a reagent to be used in the pursuit of understanding SARS-CoV-2 and the resulting COVID-19 disease. Compared with the clinical isolate, the recombinant wild-type SARS-CoV-2 has no deficit in terms of viral RNA species produced, plaque morphology, or replication kinetics. Therefore, it may be used as an equivalent to the clinical strain, and mutant viruses can be generated to characterize mutational effect on viral infection. This approach has allowed researchers to identify key viral antagonists of innate immunity for SARS-CoV and MERS-CoV through point mutations, deletions, and truncations (Nelemans and Kikkert, 2019; Totura and Baric, 2012). Several of these mutant viruses have subsequently been employed as live-attenuated vaccine candidates for SARS-CoV and MERS-CoV (de Wit et al., 2026; Schindewolf and Menachery, 2019). Using the described system, this knowledge may now be applied to the current SARS-CoV-2. The infectious clone readily allows testing and characterizing mutations. Such studies will provide insight into SARS-CoV-2 infection.

The described reverse genetic system also allows exploration of research questions fundamental to understanding the SARS-CoV-2 pandemic. As additional genomic sequences become available, evolutionary mutations can be interrogated for their effect on viral transmission and disease outcome.

A robust reverse genetic system for SARS-CoV-2 is described that can be used to study viral replication and pathogenesis. The inventors have also established an mNG reporter SARS-CoV-2 that is a reliable surrogate for high-throughput drug discovery. The reverse genetic system represents a major tool for the research community and significantly advances opportunities for countermeasure development for COVID-19.

In certain embodiments, a kit can contain nucleic acids and/or expression vectors described herein, as well as transfection and culture reagents. A standard operating procedure (SOP) can provide guidance for use of the kit. The kit system can be used for a variety of research endeavors.

### I. CORONAVIRUSES

Coronaviruses (order Nidovirales, family Coronaviridae) are a diverse group of enveloped, positive-stranded RNA viruses. The coronavirus genome, approximately 27-32 Kb in length, is the largest found in any of the RNA viruses. Large Spike (S) glycoproteins protrude from the virus particle giving coronaviruses a distinctive corona-like appearance when visualized by electron microscopy. Coronaviruses infect a wide variety of species, including canine, feline, porcine, murine, bovine, avian and human (Holmes, et al., 1996, Coronaviridae: the viruses and their replication, p. 1075-1094, Fields Virology, Lippincott-Raven, Philadelphia, Pa.). However, the natural host range of each coronavirus strain is narrow, typically consisting of a single species. Coronaviruses typically bind to target cells through Spike-receptor interactions and enter cells by receptor mediated endocytosis or fusion with the plasma membrane (Holmes, et al., 1996, supra).

Upon entry into susceptible cells, the open reading frame (ORF) nearest the 5′ terminus of the coronavirus genome is translated into a large polyprotein. This polyprotein is autocatalytically cleaved by viral-encoded proteases, to yield multiple proteins that together serve as a virus-specific, RNA-dependent RNA polymerase (RdRP). The RdRP replicates the viral genome and generates 3′ coterminal nested subgenomic RNAs. Subgenomic RNAs include capped, polyadenylated RNAs that serve as mRNAs, and antisense subgenomic RNAs complementary to mRNAs. In one embodiment, each of the subgenomic RNA molecules shares the same short leader sequence fused to the body of each gene at conserved sequence elements known as intergenic sequences (IGS), transcriptional regulating sequences (TRS) or transcription activation sequences. It has been controversial as to whether the nested subgenomic RNAs are generated during positive or negative strand synthesis; however, recent work favors the model of discontinuous transcription during minus strand synthesis (Sawicki, et al., 1995, Adv. Exp. Med. Biol. 380:499-506; Sawicki and Sawicki Adv. Expt. Biol. 1998, 440:215).

A SARS-CoV-2 reference sequence can be found in GenBank accession NC 045512.2 as of Mar. 2, 2020 (SEQ ID NO:1). This sequence is a 29903 bp ss-RNA and is referred to as the Wuhan seafood market pneumonia virus isolate Wuhan-Hu-1. The virus is Severe acute respiratory syndrome coronavirus 2 (SARS-CoV-2) with the taxonomy of Viruses; Riboviria; Nidovirales; Cornidovirineae; Coronaviridae; Orthocoronavirinae; Betacoronavirus; Sarbecovirus. (Wu et al. “A novel coronavirus associated with a respiratory disease in Wuhan of Hubei province, China” Unpublished; NCBI Genome Project, Direct Submission, Submitted (17 Jan. 2020) National Center for Biotechnology Information, NIH, Bethesda, MD 20894, USA; Wu et al. Direct Submission, Submitted (5 Jan. 2020) Shanghai Public Health Clinical Center and School of Public Health, Fudan University, Shanghai, China).

The genome of SARS-CoV-2, with reference to SEQ ID NO:1, includes (1) a 5′UTR 1-265), (2) Orflab gene (266-21555), S gene encoding a spike protein (21563 . . . 25384), ORF3a gene (25393 . . . 26220), E gene encoding E protein (26245 . . . 26472), M gene (26523 . . . 27191), ORF6 gene (27202 . . . 27387), ORF7a gene (27394 . . . 27759), ORF7b gene (27756 . . . 27887), ORF8 gene (27894 . . . 28259), N gene (28274 . . . 29533), ORF10 gene (29558 . . . 29674), and 3′UTR (29675 . . . 29903). In certain aspects, ORF7 is substituted by a nucleic acid encoding a reporter protein.

The reporter protein is a protein that can be detected, directly or indirectly, and includes colorimetric, fluorescent or luminescent proteins. Examples of luminescent or marker proteins that can be used in embodiments of the invention include, but are not limited to, Aequorin, firefly luciferase, Renilla luciferase, red luciferase, luxAB, and nanoluciferase. Examples of chemiluminescent protein or marker protein include β-galactosidase, horseradish peroxidase (HRP), and alkaline phosphatase. Examples of fluorescent protein or marker protein include, but are not limited to, mNeonGreen, TagBFP, Azurite, EBFP2, mKalama1, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOK, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKate1, LSS-mKate2, PA-GFP, PAmCherry 1, PATagRFP, Kaede (green), Kaede (red), KikGR1 (green), KikGR1 (red), PS-CFP2, PS-CFP2, mEos2 (green), mEos2 (red), PSmOrange, or Dronpa.

### II. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Design of a SARS-CoV-2 Full-Length cDNA.

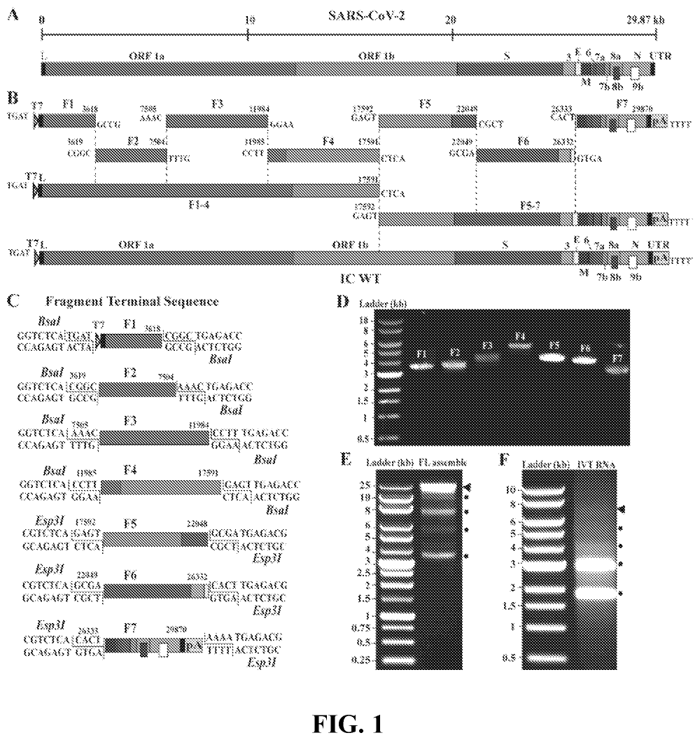

An in vitro ligation approach was used to directionally assemble the full-length cDNA of the SARS-CoV-2 genome (FIG. 1A). The reverse genetic system was based on the virus strain (2019-nCoV/USA_WA1/2020) isolated from the first reported SARS-CoV-2 case in the US (Harcourt et al., 2020; Holshue et al., 2020). Viral RNA, extracted from the passage 5 virus from Vero E6 cells, was used as a template for RT-PCR to produce cDNA fragments. Seven contiguous cDNA fragments were constructed to cover the entire viral genome (FIG. 1B). Some of the seven cDNA fragments were prepared through RT-PCR, whereas others were generated by chemical synthesis (see below for details). All cDNA fragments were individually cloned into plasmid vectors. For facilitating directional assembly of genome-length cDNA, each cDNA fragment was flanked by a class IIS restriction endonuclease site (BsaI or Esp3I). The class IIS endonucleases recognize asymmetric DNA sequences, cleave outside their recognition sequences, and generate unique cohesive overhangs (FIG. 1C). After digestion with BsaI or Esp3I, the seven fragments were directionally ligated to assemble the genome-length cDNA. The unique cohesive ends of each fragment ensured one directional, seamless assembly of the seven fragments with the concomitant loss of the restriction enzyme sites. FIG. 1C depicts the details of the seven fragments: F1 (T7 promoter sequence plus nucleotides 1-3,618), F2 (3,619-7,504), F3 (7,505-11,984), F4 (11,985-17,591), F5 (17,592-22,048), F6 (22,049-26,332), and F7 (26,333-29,870 plus a poly(A) 29 sequence). A T7 promoter and a poly(A)₂₉ tail were engineered at the upstream of F1 and the downstream of F7, respectively. In vitro transcription of the ligated F1-7 DNA was expected to produce a 5′ capped and 3′ polyadenylated genome-length RNA. To differentiate the infectious clone-derived virus from the parental clinical isolate, three synonymous nucleotide mutations were engineered as markers.

Assembly of a SARS-CoV-2 Full-Length cDNA.

Each of the seven cDNA fragments was cloned into a plasmid and sequenced to ensure no undesired mutations. For assembly of full-length cDNA, the seven cDNA plasmids were digested with BsaI or Esp3I. The resulting cDNA fragments were gel-purified (FIG. 1D), then in vitro ligated to assemble the genome-length cDNA in three steps: (i) ligation of F1, F2, F3, and F4 to produce F1-4 cDNA; (ii) ligation of F5, F6, and F7 to produce F5-7 cDNA; and (iii) ligation of F1-4 and F5-7 to produce the full-length F1-7 cDNA. Agarose gel analysis of the ligation (iii) reaction showed a major DNA product representing the size of genome-length cDNA (˜29.87 kb, indicated by an arrow in FIG. 1E) in addition to several smaller intermediate cDNA products (indicated by circles). In vitro transcription using the cDNA template [directly from ligation (iii) without gel purification] generated multiple RNA bands, among which a faint high molecular band may represent the genome-length RNA (indicated by an arrow in FIG. 1F) together with several smaller RNA transcripts (indicated by circles).

Recovery of Recombinant SARS-CoV-2.

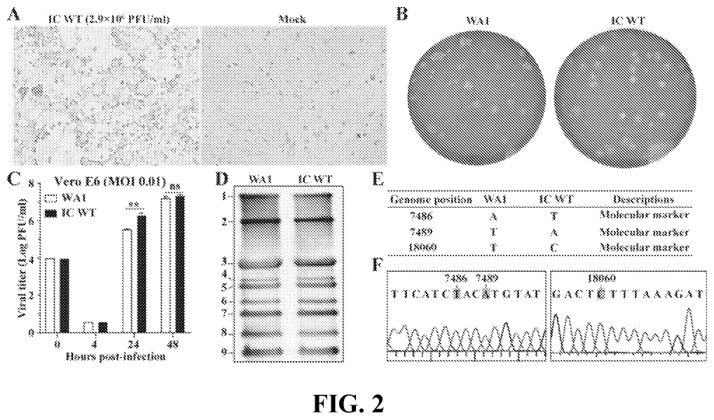

To recover recombinant SARS-CoV-2 from the infectious cDNA clone (icSARS-CoV-2), in vitro transcribed genome-length RNA was electroporated into Vero cells. The RNA transcription mixture from FIG. 1F was directly electroporated into cells without purification. Since N protein was reported to enhance the infectivity of coronavirus RNA transcripts (Curtis et al., 2002; Yount et al., 2003; Yount et al., 2002). An mRNA encoding the SARS-CoV-2 N protein was co-electroporated with the full-length RNA. The transfected cells developed cytopathic effects (CPE) on day 4 post-transfection and produced infectious virus [denoted as passage 0 (P0) virus] with a titer of 2.9×10⁶ PFU/ml (FIG. 2A). It is worth emphasizing that such a high titer of recombinant virus was produced directly from the electroporated cells without additional rounds of cell culture passaging, indicating the robustness of the system and also suggesting a lack of any errors. Next, replication properties were compared between the recombinant virus and the original clinical isolate. The wild-type icSARS-CoV-2 (icSARS-CoV-2-WT) developed plaques similar to the original clinical isolate (FIG. 2B) and exhibited equivalent replication kinetics on Vero cells (FIG. 2C). The time points of replication were not extended beyond 48 h because CPE was observed at 40-48 h post-infection (p.i.). Northern blot analysis showed that viral mRNA species from the clinical isolate-infected cells and the icSARS-CoV-2-infected cells were identical to the predicted set of genome-length RNA and eight subgenomic RNAs (FIG. 2D). Full-genome sequencing showed that the recombinant virus retained the three engineered synonymous mutations with no other sequence changes, demonstrating the rescued virus did not result from contamination by the parental virus isolate (FIG. 2E). Furthermore, DNA sequencing chromatogram did not show any partial reversion of the three engineered molecular markers (FIG. 2F). Collectively, the results demonstrate that (i) the in vitro transcribed full-length RNA is highly infectious upon electroporation into cells and (ii) the recombinant virus recapitulates the replication properties of the original clinical isolate on Vero cells.

Development and Characterization of mNeonGreen SARS-CoV-2.

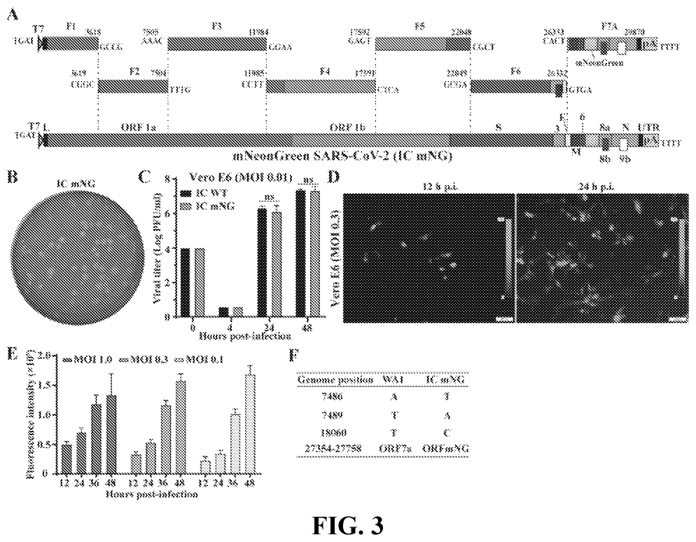

Reporter viruses are useful tools to study viral replication and pathogenesis and to develop countermeasure. To establish a reporter SARS-CoV-2 infectious clone, an mNeonGreen (mNG) gene was engineered into the ORF7 of viral genome (FIG. 3A), similar to the SARS-CoV reporter (Sims et al., 2005). The same in vitro ligation and transcription protocols (described above) were used to prepare the mNG full-length RNA. After electroporation, icSARS-CoV-2-mNG (6.9×10⁶ PFU/ml) was recovered. To examine if the reporter gene attenuates viral replication, the replication properties were compared between the wild-type and reporter viruses on Vero E6 cells. The icSARS-CoV-2-mNG produced plaques similar to those of the icSARS-CoV-WT (compare FIG. 3B with FIG. 2B). Indistinguishable replication kinetics were observed for the icSARS-CoV-2-mNG and icSARS-CoV-WT (FIG. 3C). Infection with icSARS-CoV-2-mNG developed increasing numbers of mNG-positive cells over time (FIG. 3D). Concurrently, the fluorescent signals increased from 12 to 48 h p.i. (FIG. 3E). At 12-36 h p.i., the level of fluorescent signal correlated with the initial MOIs, whereas a reverse trend was observed at 48 h p.i., most likely due to earlier CPE caused by the higher MOI. Full-genome sequencing showed that icSARS-CoV-2-mNG retained the three engineered markers with no additional mutations (FIG. 3F). These results indicate that icSARS-CoV-2-mNG is initially stable, maintains the wild-type replication, and expresses robust mNG in Vero E6 cells.

Stability of icSARS-CoV-2-mNG.

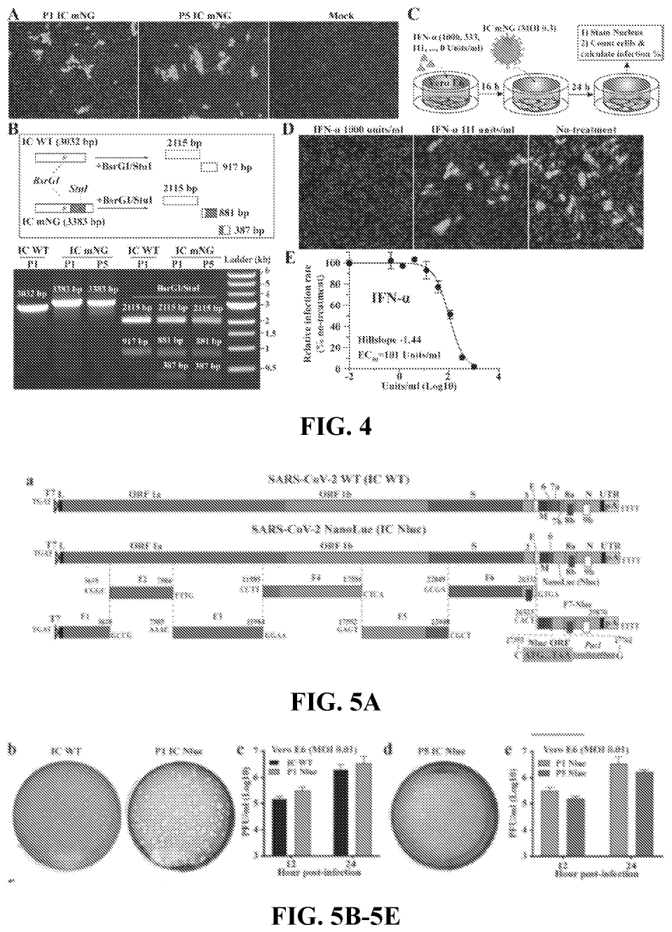

To examine the longer-term stability of icSARS-CoV-2-mNG, the reporter virus was serially passaged on Vero cells for 5 rounds (1 to 2 days per round). Cells infected with equal PFU of passage 1 (P1) or passage 5 (P5) viruses produced comparable numbers of mNG-positive cells (FIG. 4A). Next, RT-PCR was performed to verify the retention of mNG in the P1 and P5 viral genomes using two primers targeting the insertion junctions (corresponding to nucleotides 25,068-28,099 of the viral genome). As expected, the RT-PCT products derived from both P1 and P5 mNG viruses were larger than those from the wild-type icSARS-CoV-2 (FIG. 4B, lanes 1-3). Digestion of the RT-PCR products with BsrGI (located upstream of the mNG insertion site) and StuI (in the mNG gene) developed distinct cleavage patterns between the reporter and wild-type viruses, whereas P1 and P5 viruses produced an identical digestion pattern (FIG. 4B, lanes 4-6). Finally, sequencing the P1 and P5 RT-PCR products confirmed the retention of the mNG gene (data not shown). Altogether, the results demonstrate the stability of icSARS-CoV-2-mNG after five rounds of passaging on Vero cells.

Application of icSARS-CoV-2-mNG.

To explore the utility of icSARS-CoV-2-mNG, the reporter virus was used to rapidly screen the antiviral activity of a known inhibitor of coronaviruses. It was previously showed that pre-treatment of Vero cells with type-I interferon (IFN) inhibits SARS-CoV-2 replication (Lokugamage et al., 2020). Here the dose responsive effect of IFN-α pre-treatment on icSARS-CoV-mNG replication was explored (FIG. 4C). No mNG expression was visually observed when the infected cells were pre-treated with the highest dose of IFN-α (1,000 u/ml), whereas a dose-dependent reduction of mNG signal was detected at an intermediate dose (111 u/ml) (FIG. 4D). Quantification of the fluorescent readouts estimated an EC₅₀ (concentration inhibiting 50% of viral replication) of 101 u/ml, confirming the inhibition of SARS-CoV-2 by IFN-α (FIG. 4E). This result is consistent with previous findings that SARS-CoV-2 is sensitive to type-I IFN inhibition. The reporter virus assay required fewer days and less labor when compared with the conventional plaque reduction assay. Collectively, the results indicate that icSARS-CoV-2-mNG could be reliably used to study SARS-CoV-2 replication and to screen antiviral inhibitors.

### Example 2

### A Nanoluciferase SARS-CoV-2 for Rapid Neutralization Testing and Screening of Anti-Infective Drugs for COVID-19

A high-throughput platform would greatly facilitate COVID-19 serological testing and antiviral screening. Here it is reported a nanoluciferase SARS-CoV-2 (SARS-CoV-2-Nluc) that is genetically stable and replicates similarly to the wild-type virus in cell culture. The inventors demonstrate that the optimized reporter virus assay in Vero E6 cells can be used to measure neutralizing antibody activity in patient sera and produces results in concordance with a plaque reduction neutralization test (PRNT). Compared with the low-throughput PRNT (3 days), the reporter virus assay has substantially shorter turnaround time (5 hours) with a high-throughput testing power. Thus, the assay can be readily deployed for large-scale vaccine evaluation and neutralizing antibody testing in humans. Additionally, the inventors developed a high-throughput antiviral assay using the SARS-CoV-2-Nluc infection of A549 cells expressing human ACE2 receptor (A549-hACE2). When tested against this reporter virus, remdesivir exhibited substantially more potent activity in A549-hACE2 cells compared to Vero E6 cells (EC₅₀ 0.115 vs 1.28 while this difference was not observed for chloroquine (EC₅₀ 1.32 vs 3.52 underscoring the importance of selecting appropriate cells for antiviral testing. Using the optimized SARS-CoV-2-Nluc assay, the inventors evaluated a collection of approved and investigational antivirals and other anti-infective drugs. Nelfinavir, rupintrivir, and cobicistat were identified as the most selective inhibitors of SARS-CoV-2 (EC₅₀ 0.77 to 2.74 In contrast, most of the evaluated clinically approved antivirals including tenofovir alafenamide, emtricitabine, sofosbuvir, ledipasvir, and velpatasvir were inactive. Collectively, this high-throughput assay platform represents a reliable tool for rapid neutralization testing and antiviral screening for SARS-CoV-2.

### Results

A Stable SARS-CoV-2-Nluc.

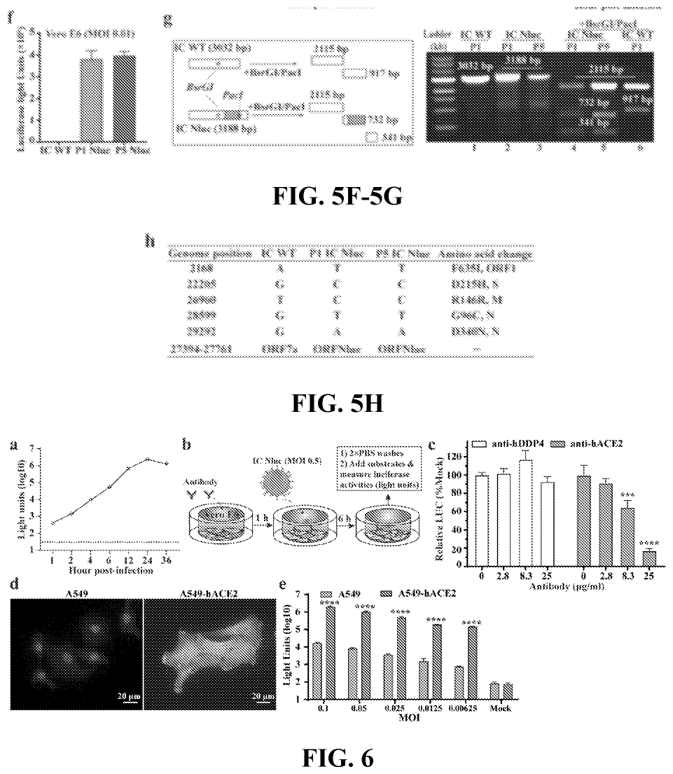

Using an infectious cDNA clone of SARS-CoV-2 (strain 2019-nCoV/USA WA1/2020), the inventors engineered nanoluciferase (Nluc) gene at the OFR7 of the viral genome (FIG. 5A). Seven cDNA fragments spanning the SARS-CoV-2 genome were in vitro ligated to generate a full-genome Nluc cDNA. A T7 promoter was engineered to in vitro transcribe the full-length Nluc viral RNA. The RNA transcript was highly infectious after electroporation into Vero E6 cells (African green monkey kidney epithelial cells), producing 10⁷ PFU/ml of virus. The infectious clone-derived SARS-CoV-2-Nluc developed plaques slightly larger than the wild-type recombinant SARS-CoV-2 (FIG. 5B). The SARS-CoV-2-Nluc and wild-type SARS-CoV-2 exhibited similar replication kinetics in Vero E6 cells (FIG. 5C), indicating that insertion of Nluc gene does not affect viral replication in vitro.

To examine the stability of SARS-CoV-2-Nluc, the inventors continuously cultured the virus for five passages in Vero E6 cells (1-2 days per passage). Compared with passage 1 (P1) SARS-CoV-2-Nluc, P5 virus produced similar plaque morphology (FIG. 5D), replication kinetics (FIG. 5E), and luciferase profile (FIG. 5F). Next, RT-PCR was performed to verify the retention of Nluc gene in the P1 and P5 viral genomes using two primers spanning the insertion junctions (nucleotides 25,068-28,099 of viral genome). The RT-PCR products derived from both P1 and P5 Nluc viruses were 156-bp larger than that from the wild-type recombinant SARS-CoV-2 (FIG. 5G, lanes 1-3). The 156-bp difference is due to the substitution of ORF7 (368 bp) with Nluc gene (513 bp). Digestion of the RT-PCR products with BsrGI (located upstream of the Nluc insertion) and Pad (located at the C-terminal region of Nluc) generated distinct DNA fragments between the Nluc and wild-type viruses, whereas the P1 and P5 viruses produced identical digestion patterns (FIG. 5G, lanes 4-6). Furthermore, the inventors confirmed the retention of Nluc reporter by sequencing the P1 and P5 RT-PCR products (FIG. 5H). Compared with the infectious clone-derived wild-type SARS-CoV-2, both P1 and P5 reporter viruses contained five single nucleotide mutations, which led to amino acid changes in different viral proteins (FIG. 5H). These mutations may account for the slightly larger plaques of SARS-CoV-2-Nluc. No other mutations were recovered from the passaged viruses. Altogether, the results demonstrate that SARS-CoV-2-Nluc is stable after five rounds of passaging on Vero E6 cells.

Human Angiotensin-Converting Enzyme (hACE2) as a Receptor for SARS-CoV-2.

The inventors explored SARS-CoV-2-Nluc to study virus entry, serological diagnosis, and antiviral screening. Infection of Vero E6 cells with SARS-CoV-2-Nluc [multiplicity of infection (MOI) 1.0] produced a robust Nluc profile that peaked at 24 h post infection (p.i.; FIG. 6A). As early as 1 h p.i., the Nluc signal was >10 fold above the background, suggesting that Nluc signals at early timepoints may be used to study virus entry. Thus, the function of hACE2 in virus entry was calculated by pre-incubating Vero E6 cells with anti-hACE2 polyclonal antibodies for 1 h, followed by SARS-CoV-2-Nluc infection (FIG. 6B). The anti-hACE2 antibodies inhibited Nluc signal at 6 h p.i. in a dose-responsive manner (FIG. 6C). As a negative control, pre-treatment with antibodies against hDPP4 (a receptor for MERS-CoV infection) did not suppress Nluc activity (FIG. 6C), indicating the role of hACE2 in SARS-CoV-2 entry. To further evaluate these results, the efficiencies of virus entry was compared between naïve A549 (a human alveolar epithelial cell line) and A549 stably expressing hACE2 (A549-hACE2; FIG. 6D). At various MOIs, the Nluc signals (collected at 24 h p.i.) from A549-hACE2 cells were ˜100-fold higher than those from naïve A549 cells (FIG. 5E). Collectively, the results support hACE2 as a receptor for SARS-COV-2 entry.

A Rapid Neutralization Assay for COVID-19 Diagnosis.

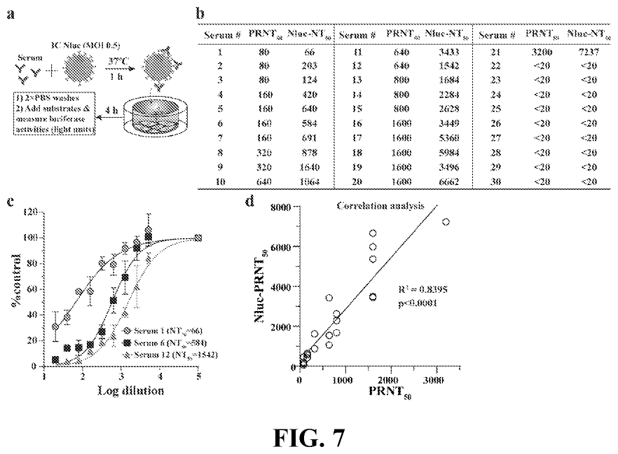

The robust early Nluc signals after SARS-COV-2-Nluc infection (FIG. 6A) prompted us to develop a rapid neutralization assay. FIG. 7A depicts the flowchart of SARS-COV-2-Nluc neutralization assay in a 96-well format. After incubating serum samples with SARS-COV-2-Nluc at 37° C. for 1 h, the virus/serum mixtures were added to Vero E6 cells (pre-seeded in a 96-well plate) at an MOI of 0.5. At 4 h p.i., Nluc signals were measured to determine the serum dilution fold that neutralized 50% of Nluc activity (NT₅₀). The assay end time was chosen as 4 h p.i. because the Nluc signal at this timepoint was >100 fold above the background (FIG. 6A). The total assay time to completion was 5 h (1 h virus/serum incubation plus 4 h viral infection). Following this protocol, twenty-one COVID-19-positive sera from RT-PCR-confirmed patients and nine COVID-19-negative human sera (collected before COVID-19 emergence; FIG. 7B) were tested. All COVID-19-positive sera (samples 1-21) showed positive NT₅₀ of 66 to 7237, while all COVID-19-negative sera (samples 22-30) showed negative NT₅₀<20, the lowest tested serum dilution. FIG. 7C shows three representative neutralization curves: Nluc signals were suppressed by the positive sera in an inverse dilution-dependent manner. The results suggest that SARS-COV-2-Nluc could be used for rapid neutralization testing.

To validate the Nluc neutralization results, conventional PRNT was performed on the same set of patient sera. The twenty-one COVID-19-positive samples exhibited PRNT₅₀ of 80 to 3200, and the nine COVID-19-negative samples showed PRNT₅₀<20 (FIG. 7B). The neutralization results between the Nluc virus and PRNT assays had a correlation coefficient (R²) of 0.8395 (FIG. 7D). Notably, the NT₅₀ values from the Nluc assay are on average 3-fold higher than the PRNT₅₀ values form the plaque assay. Overall, the results indicate that the SARS-CoV-2-Nluc neutralization assay detects neutralizing antibodies in COVID-19 patient sera with a higher sensitivity than the conventional PRNT assay.

A High-Throughput Antiviral Assay for SARS-CoV-2.

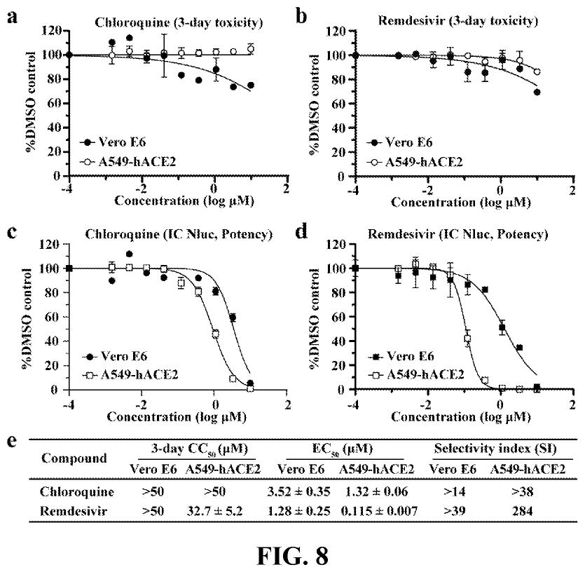

Reporter viruses have been commonly used for antiviral screening (Puig-Basagoiti et al. Antimicrob. Agent. Chemother. 49, 4980-4988, 2005; Zou et al. Antiviral Res 91, 11-19, 2011; Shan et al. Cell Host Microbe 19, 891-900, 2016; Scobey et al. Proc Natl Acad Sci USA 110, 16157-16162, 2013; Almazan et al. Virus Res 189, 262-270, 2014; Hou et al. Cell 182, 1-18, 2020; Roberts et al. Adv Exp Med Biol 581, 597-600, 2006). Therefore, a 96-well format antiviral assay was developed using the SARS-CoV-2-Nluc reporter virus. Vero E6 cells were initially used because this cell line is highly susceptible to SARS-CoV-2 infection (Zhou et al. Nature 579, 270-273, 2020). Since COVID-19 is a respiratory disease, A549 (a human alveolar epithelial cell line) were also tested for assay development. However, due to the low permissiveness of A549 for SARS-CoV-2-Nluc infection, A549-hACE2 cells were included to enhance viral infection in the assay (FIG. 6E). Two SARS-CoV-2 inhibitors that received the emergency use authorization in US for COVID-19 at the time of assay development, chloroquine phosphate (a malaria drug) and remdesivir (an antiviral adenosine analog prodrug)(Wang et al. Cell Res 30, 269-271, 2020), were used to evaluate the assay in both Vero E6 and A549-hACE2 cells (FIG. 8). In a 3-day cytotoxicity assay, chloroquine showed CC₅₀ of >50 μM on both cells, whereas remdesivir had CC₅₀ of >50 μM and 32.5 μM in Vero E6 and A549-hACE2 cells, respectively (FIG. 8A, 8B). For testing of the antiviral activity, the assay conditions (12,000 cells per well and MOI 0.025) were optimized to allow for multiple rounds of viral replication in 48 h p.i. without developing significant cytopathic effect (CPE). Both chloroquine and remdesivir inhibited Nluc activity in a dose-dependent manner (FIG. 8C, 8D). Importantly, the EC₅₀ values for remdesivir in A549-hACE2 cells (0.115 μM) were >10-fold lower than those in Vero E6 cells (1.28 μM), while the potency of chloroquine was only marginally different between the two cell lines (EC₅₀ 1.32 vs 3.52 μM; FIG. 8E). This result underscores the importance of using biologically relevant cells for antiviral testing. Thus, A549-hACE2 were chosen for the following high-throughput antiviral screening of additional compounds.

Testing of Clinically Relevant Anti-Infective Drugs for Antiviral Activity Against SARS-CoV-2.

A broad selection of forty clinically approved and investigational antivirals and other anti-infective drugs were tested for anti-SARS-CoV-2-Nluc activities in A549-hACE2 cells. Based on their indication and/or mode of action, the tested drugs belong to four categories, including (i) antiviral nucleoside/nucleotide analogs, (ii) HIV antivirals, (iii) HCV antivirals, and (iv) other primarily anti-infective drugs.

(i) Nucleoside/Nucleotide Analog Drugs.

Ten nucleoside analogs with antiviral activities against other viruses were evaluated for activity against SARS-CoV-2-Nluc (Table 1). Only remdesivir showed SARS-CoV-2-Nluc activity with an EC₅₀ and CC₅₀ of 0.115 and 32.7 μM, respectively, and selectivity index (SI=CC₅₀/EC₅₀) of 284. No other nucleoside analogs, including sofosbuvir or any other 2′C-methyl substituted anti-HCV nucleosides or their prodrugs, exhibited any anti-SARS-CoV-2 activity at concentrations up to 10 μM. The results agree with previous reports demonstrating potent inhibition of SARS-CoV-2 by remdesivir in physiologically relevant airway epithelial cells¹³, and lack of SARS-CoV-2 inhibition by favipiravir and/or ribavirin (Choy et al. Antiviral Res 178, 104786, 2020; Jeon et al. Antimicrob Agents Chemother, 2020; Liu et al. Nucleosides Nucleotides Nucleic Acids 31, 277-285, 2012).

(ii) HIV Antivirals.

Sixteen clinically approved antiretrovirals, including protease inhibitors (PIs), nucleoside/nucleotide reverse-transcriptase inhibitors (NRTIs), non-nucleoside reverse transcriptase inhibitors (NNRTIs), and integrase strand-transfer inhibitors (INSTIs), were assessed for their activities against SARS-CoV-2-Nluc (Table 2). Among the nine FDA-approved HIV PIs tested, nelfinavir was the only compound that inhibited SARS-CoV-2-Nluc with a sub-micromolar potency (EC₅₀ 0.77 μM), albeit with a relatively narrow SI of 16. Factoring in human plasma protein binding of nelfinavir (Molla et al. Virology 250, 255-262, 1998), the projected protein adjusted potency (paEC₅₀˜30 μM) is significantly above the clinically achievable plasma concentration of the drug (Table 2). Of the remaining PIs, five were inactive (amprenavir, ritonavir, indinavir, darunavir, and atazanavir) and three exhibited rather weak antiviral activity (lopinavir, saquinavir, and tipranavir with EC₅₀ of 8-9 μM and SI of 3-4).

Among the HIV RT inhibitors, all three NRTIs (emtricitabine, tenofovir alafenamide, and rovafovir) were inactive against SARS-CoV-2-Nluc with EC₅₀>10 μM (Table 2). The two NNRTIs (rilpivirine and efavirenz) exhibited poor SI<3.9. Bictegravir, a drug targeting HIV integrase, was inactive against SARS-CoV-2-Nluc with EC₅₀>10 μM (Table 2).

(iii) HCV Antivirals.

Nine FDA-approved HCV drugs with diverse modes of action targeting viral protease, polymerase (both nucleotide and non-nucleoside inhibitors), or NS5A protein were tested. None of them showed any anti-SARS-CoV-2-Nluc activities with EC₅₀>10 μM (Table 3).

(iv) Other Classes of Drugs.

Ten additional clinically validated drugs, six of which are anti-infective medicines, were tested against SARS-CoV-2-Nluc (Table 4). Rupintrivir, a human rhinovirus (HRV) 3CLpro cysteine protease inhibitor, inhibited SARS-CoV-2-Nluc with EC₅₀ 1.87 μM, representing a 156-fold lower potency than that against HRV¹⁸. Niclosamide (an antihelminthic drug) showed an anti-SARS-COV-2-Nluc activity (EC₅₀ 0.715 μM) with low selectivity (SI 1.8). As described in FIG. 8, chloroquine exhibited selective inhibition of anti-SARS-CoV-2-Nluc (EC₅₀ 1.32 μM and SI>37.9). Presatovir, a respiratory syncytial virus (RSV) fusion inhibitor, showed an EC₅₀ of 2.53 μM and SI of >37.9. The EC₅₀ of presatovir against SARS-CoV-2 is 7,000-fold less potent than against RSV (Perron et al. Antimicrob Agents Chemother 60, 1264-1273, 2015), precluding the potential for COVID-19 therapy. Cobicistat, a selective mechanism-based inhibitor of CYP3A enzymes, weakly inhibited SARS-CoV-2-Nluc (EC₅₀ 2.7 μM) and with a modest SI of 17.3. Oseltamavir carboxylate and baloxavir, two approved drugs targeting influenza A virus neuraminidase and endonuclease, respectively, were inactive against SARS-CoV-2-Nluc with EC₅₀>10 μM. Nivocasan, an inhibitor of cellular caspases 1, 8, and 9 (treatment for hepatic fibrosis and non-alcoholic steatohepatitis related to HCV infection), as well as two inhibitors of Bruton's tyrosine kinase (BTK; treatment for lymphoma and leukemia) were also inactive against SARS-CoV-2 (Table 4).

Taken together, the results indicate that, among the forty clinically validated and investigational anti-infective drugs tested, only remdesivir, chloroquine, and rupintrivir have the potential for COVID-19 therapy.

A stable reporter SARS-CoV-2-Nluc variant was developed for rapid neutralization testing. Since neutralizing titer is a key parameter to predict immunity, the rapid SARS-CoV-2-Nluc neutralization assay will enable many aspects of COVID-19 research, including epidemiological surveillance, vaccine development, and antiviral discovery. Although the current assay was performed in a 96-well format, given the magnitude and dynamic range of Nluc signal, it can be readily adapted to a 384- or 1536-well format for large-scale testing. Notably, when diagnosing patient sera, the SARS-CoV-2-Nluc assay generated NT₅₀ value on average 3-fold higher than the conventional PRNT₅₀. The higher sensitivity of the SARS-CoV-2-Nluc assay might be due to the amplification nature of luciferase enzyme that was used for assay readout. Compared with the conventional PRNT assay, the reporter neutralization test has shortened the turnaround time from 3 days to 5 h and increased the testing capacity.

The inventors optimized and validated the SARS-CoV-2-Nluc for a high-throughput antiviral screening. Results demonstrated that cell type could significantly affect a compound's EC₅₀ value, underscoring the importance of using biologically relevant cells for drug discovery. The extent of EC₅₀ discrepancy from different cells was dependent on the compound's mode of action. When remdesivir was tested on Vero E6 and A549-hACE2 cells, the EC₅₀ values differed by >10-fold. In another study, remdesivir was shown to be even more potent (EC₅₀ 0.01 μM) when tested on primary human airway epithelial (HAE) cells (Pruijssers et al. bioRxiv 2020). The differences seen between cell types are due to the differential metabolism of remdesivir in the various cells. Host metabolic enzymes are required to convert the remdesivir prodrug to a monophosphate substrate, which is further metabolized by host kinases to its active triphosphate form that incorporates into viral RNA for chain termination. Vero E6 cells are less efficient in forming the active triphosphate than A549-hACE2 and primary HAE cells (Pruijssers et al. bioRxiv 2020; Gordon et al. J Blot Chem 295, 6785-6797, 2020), leading to higher EC₅₀ values. The antiviral activity of chloroquine was more consistent between the two cell lines tested, indicating that its mode of action is independent of host metabolism. This underscores the need for careful and appropriate interpretation of in vitro antiviral data for compounds with different mechanisms of action such as remdesivir and chloroquine, which may appear similar in some cell types but are substantially different in cells that are more clinically relevant for SARS-CoV-2 infection. Remdesivir has received the FDA's emergency use authorization for COVID-19 treatment and is being tested in additional clinical trials, including combination therapies. In a double-blind, randomized, placebo-controlled trial involving 1,063 patients hospitalized with COVID-19, patients receiving remdesivir experienced a shortened recovery time of 11 days as compared with 15 days for patients in the placebo group (Beigel et al. N Engl J Med, 2020). Besides SARS-CoV-2, remdesivir was also shown to potently inhibit SARS-CoV and MERS-CoV in cell culture and animal models (Pruijssers et al. bioRxiv 2020; de Wit et al. Proc Natl Acad Sci USA 117, 6771-6776, 2020; Williamson et al. bioRxiv 2020; Sheahan et al. Nat Commun 11, 222, 2020; Sheahan et al. Sci Transl Med 9, 2017. For chloroquine, inconsistent results were obtained from several clinical studies with small patient numbers (Gao et al. Biosci Trends 14, 72-73, 2020; Gautret et al. Travel Med Infect Dis 34, 101663, 2020; Molina et al. Med Mal Infect 2020). A recent retrospective multicenter study involving >1,400 patients showed that treatment with hydroxychloroquine, azithromycin, or both, compared with no treatment, was not associated with significant differences in fatality rate among hospitalized patients (Rosenberg et al. JAMA, 2020).

Using the validated SARS-CoV-2-Nluc/A549-hACE2 infection assay, the inventors screened a collection of almost 40 clinically relevant antivirals and anti-infective drugs. In addition to remdesivir and chloroquine used for the assay validation, nelfinavir (HIV protease inhibitor), rupintrivir (HRV protease inhibitor), and cobicistat (a pharmacoenhancer and inhibitor of CYP450) were identified as the most potent and selective inhibitors among the tested compounds with EC₅₀ values ranging from 0.77 to 2.74 μM and SI>15-fold. In studies with HIV, a 40-fold shift in the antiviral EC 50 was reported when assays were conducted in the presence of 50% human serum (Molla et al. Virology 250, 255-262, 1998), an effect likely relevant for COVID-19 as well. Based on their established potencies, it is unlikely that nelfinavir or cobicistat would exert major clinical effects in COVID-19 patients at the current clinically approved doses, since their systemic free drug levels based on total plasma concentration and established plasma protein binding are below their measured in vitro EC₅₀ for SARS-CoV-2-Nluc (Siegel et al. J Med Chem 60, 1648-1661, 2017; De Clercq, Clin Microbiol Rev 16, 569-596, 2003). Rupintrivir is a selective covalent inhibitor of HRV 3CLpro cysteine protease (Kawatkar et al. Bioorg Med Chem Lett 26, 3248-3252, 2016), and thus may inhibit SARS-CoV-2 through blocking the main 3CLpro cysteine protease activity. Rupintrivir has a potent activity in vitro against HRV that is approximately 100-fold better compared to SARS-CoV-2 (Patick et al. Antimicrob Agents Chemother 43, 2444-2450, 1999). It has been tested clinically as an intranasal spray for the treatment of HRV-associated common cold (Hayden et al. Antimicrob Agents Chemother 47, 3907-3916, 2003), but there is no clinical experience with either systemic or inhaled administration of rupintrivir. Hence, further studies would be required to better understand rupintrivir's mode of action, efficacy in animal models, and potential clinical benefits in COVID-19 patients depending on the route of administration. Several antiviral drugs approved for the treatment of HIV or HCV have been suggested to be potentially useful for the treatment of COVID-19 (Copertino et al. ChemRxiv 2020; Elfiky, Life Sci 253, 117592, 2020). These include particularly sofosbuvir either alone (Elfiky, Life Sci 253, 117592, 2020; Jácome et al. Sci Rep 10, 2020) or in combination with velpatasvir (Izzi et al. Eur Rev Med Pharmacol Sci 24, 5193-5194, 2020), tenofovir (Chien et al. bioRxiv 2020), and emtricitabine (Copertino et al. ChemRxiv 2020; Elfiky, Life Sci 253, 117592, 2020). Their activities against SARS-CoV-2 were postulated primarily based on computational modeling of their interactions with the viral RdRp. The results clearly demonstrate the lack of antiviral activity of this group of drugs against SARS-CoV-2; therefore, these drugs do not justify clinical studies in COVID-19 patients.

TABLE 1
Nucleoside and nucleotide analogs against SARS-CoV-2-Nluc

| Compound | | | | |
|---|---|---|---|---|
| name | EC₅₀ (μM)^{a} | CC₅₀ (μM)^{a} | SI^{b} | Nucleoside analog |
| Remdesivir | 0.115 ± 0.007 | 32.7 ± 5.2 | 284 | 1′-CN-C-adenosine |
| (GS-5734) | | | | |
| GS-6620 | >10 | >50 | — | 1′CN, 2′Me-C- |
| | | | | adenosine |
| MK-0608 | >10 | >50 | — | 2′Me-7-deaza- |
| | | | | adenosine |
| PSI-352938 | >10 | >50 | — | 2′Me-2′F-guanosine |
| Sofosbuvir | >10 | >50 | — | 2′Me, 2′F-uridine |
| ALS-8112 | >10 | >50 | — | 2′F, 4′Cl-Me-cytidine |
| Entecavir | >10 | >50 | — | Carbocyclic |
| | | | | deoxyguanosine |
| Cidofovir | >10 | >50 | — | Acyclic cytidine |
| Favipiravir | >10 | >50 | — | Modified nucleobase |
| (T-705) | | | | |
| Ribavirin | >10 | >50 | — | Ribofuranosyl |

ªValues are mean ± standard deviation of two independent replicates in A549-hACE2 cells
^{b}Selectivity index (SI) = CC₅₀/EC₅₀

TABLE 2
HIV drugs against SARS-CoV-2-Nluc

| Inhibitor | Compound | EC₅₀ | | | Exposure | Plasma protein |
|---|---|---|---|---|---|---|
| class | name | (μM)^{a} | CC₅₀ (μM)^{a} | SI^{b} | (μM)^{c} | binding (%)^{d} |
| HIV | Lopinavir | 9.00 ± 0.42 | 31.5 ± 2.5 | 3.5 | 15.6/8.8 | 98-99 |
| protease | Amprenavir | >10 | >50 | — | — | 90 |
| (aspartyl) | Nelfinavir | 0.77 ± 0.32 | 12.0 ± 1.3 | 15.7 | 8.3/2.6 | >98 |
| | Ritonavir | >10 | 36.9 ± 1.7 | — | — | 98-99 |
| | Indinavir | >10 | >50 | — | — | 61 |
| | Saquinavir | 8.95 ± 0.31 | 35.1 ± 11.7 | 3.9 | 3.7/0.65 | 98 |
| | Darunavir | >10 | >50 | — | — | 95 |
| | Atazanavir | >10 | >50 | — | — | 86 |
| | Tipranavir | 8.65 ± 0.16 | 28.4 ± 0.5 | 3.3 | 130/30.8 | 99.9 |
| HIV | Emtricitabine | >10 | >50 | — | Cₘₐₓ 7.9 | 4 |
| NRTI | (FTC) | | | | | |
| | Tenofovir | >10 | >50 | — | Cₘₐₓ 0.4 | 80 |
| | alafenamide | | | | | |
| | (TAF) | | | | | |
| | Rovafovir | >10 | >50 | — | — | 58 |
| | (GS-9131) | | | | | |
| HIV | Rilpivirine | 7.80 ± 1.04 | 14.6 ± 1.6 | 1.9 | 0.83/0.30 | 99.7 |
| NNRTI | Efavirenz | >9.6 | 37.6 ± 10.7 | <3.9 | 12.9/5.6 | 99.5-99.8 |
| HIV | Bictegravir^{t} | >10 | >50 | — | — | >99 |
| integrase | | | | | | |

^{a}Values are mean ± standard deviation of two independent replicates in A549-hACE2 cells
^{b}SI = CC₅₀/EC₅₀
^{c}Values represent Cₘₐₓ and Cₘᵢₙ for human exposures in the clinics
^{d}Data from literature as cited

TABLE 3
HCV drugs against SARS-COV-2-Nluc

| Inhibitor class | Compound name | EC₅₀ (μM)^{a} | CC₅₀ (μM)^{a} |
|---|---|---|---|
| HCV protease | GS-9256 | >10 | 31.8 ± 10.9 |
| (serine) | GS-9451 | >10 | >50 |
| | Voxilaprevir | >10 | 16.0 ± 1.2 |
| HCV nucleoside | Sofosbuvir | >10 | >50 |
| RdRp | | | |
| HCV non- | GS-9130 | >10 | >50 |
| nucleoside RdRp | Tegobuvir | >10 | 17.9 ± 3.1 |
| | Radalbuvir | >10 | >50 |
| HCV NS5A | Ledapisvir | >10 | >50 |
| | Velpatasvir | >10 | >50 |

ªValues are mean ± standard deviation of two independent replicates in A549-hACE2 cells

TABLE 4
Other drug classes against SARS-COV-2-Nluc

| Inhibitor class | Compound name | EC₅₀ (μM)^{a} | CC₅₀ (μM)^{a} | SI^{b} |
|---|---|---|---|---|
| HRV protease | Rupintrivir | 1.87 ± 0.47 | >50 | >26.7 |
| (serine) | | | | |
| Antihelminthic | Niclosamide | 0.715 ± 0.332 | 1.28 ± 0.23 | 1.8 |
| Antimalarial/ | Chloroquine | 1.32 ± 0.36 | >50 | >37.9 |
| amebicide | | | | |
| RSV fusion | Presatovir | 2.53 ± 0.69 | 34.0 ± 6.5 | 13.5 |
| CYP3A inhibitor | Cobicistat | 2.74 ± 0.20 | 47.3 ± 2.5 | 17.3 |
| Influenza | Oseltamivir | >10 | >50 | — |
| neuraminidase | carboxylate | | | |
| Influenza | Baloxavir | >10 | 47.0 ± 1.3 | — |
| endonuclease | | | | |
| Caspases 1, 8, & 9 | Nivocasan | >10 | >50 | — |
| | (GS-9450) | | | |
| BTK | Tirabrutinib | >10 | >50 | — |
| | Ibrutinib | >10 | >50 | — |

^{a}Values are mean ± standard deviation of two independent replicates in A549-hACE2 cells
^{b}SI = CC₅₀/EC₅₀

### Materials and Methods

Cell lines. African green monkey kidney epithelial cells Vero E6 (ATCC®CRL-1586) and Vero cell line (ATCC®CCL-81) were purchased from the American Type Culture Collection (ATCC, Bethesda, MD) and maintained in a high-glucose Dulbecco's modified Eagle's medium (DMEM) supplemented with 10% fetal bovine serum (FBS; HyClone Laboratories, South Logan, UT) and 1% penicillin/streptomycin (P/S). Human alveolar epithelial cell line (A549) and human embryonic kidney cells (HEK293) were maintained in a high-glucose DMEM supplemented with 10% fetal bovine serum, 1% P/S and 1% HEPES (ThermoFisher Scientific). The A549-hACE2 and HEK293-hACE2 cells that stably express human angiotensin-converting enzyme 2 (hACE2)⁷⁷ were grown in the culture medium supplemented with 10 μg/ml Blasticidin S. Cells were grown at 37° C. with 5% CO₂. All culture medium and antibiotics were purchased from ThermoFisher Scientific (Waltham, MA). All cell lines were tested negative for mycoplasma.

Generation of SARS-CoV-2-Nluc.

A subclone (F7-Nluc) was constructed by substituting the ORF7 of the viral genome with the reporter Nano R luciferase gene followed by a PacI restriction site (taattaattaa). All subclones were validated by Sanger sequencing prior to assembling the full-length clone. The full-length infectious cDNA clone of SARS-CoV-2-Nluc was generated by in vitro ligation of seven contiguous panel of cDNA according to a protocol as reported previously (Xie et al. Cell Host Microbe, 2020). RNA transcript was in vitro synthesized by the mMES SAGE mMACHINE™ T7 Transcription Kit (ThermoFisher Scientific) and electroporated into Vero E6 cells to recover the recombinant SARS-CoV-2-Nluc by using the same protocol as described reviously (Xie et al. Cell Host Microbe, 2020). The viral stock was prepared by amplifying the SARS-CoV-2-Nluc on Vero E6 cells for one round (P1). The titer of the virus stock was determined by a standard plaque assay. All SARS-CoV-2-Nluc propagation and other virus-related work were performed at the BSL-3 facility at UTMB.

RNA Extraction, RT-PCR and Sanger Sequencing.

250 μl of culture fluids were mixed with three volume of TRIzol™ LS Reagent (Thermo Fisher Scientific). Viral RNAs were extracted per manufacturer's instructions. The extracted RNAs were dissolved in 30 μl nuclease-free water. 11 μl RNA samples were used for reverse transcription by using the SuperScript™ IV First-Strand Synthesis System (ThermoFisher Scientific) with random hexamer primers. Nine DNA fragments flanking the entire viral genome were amplified by PCR with specific primers. The resulting DNAs were cleaned up by the QIAquick PCR Purification Kit, and the genome sequences were determined by Sanger sequencing at GENEWIZ (South Plainfield, NJ).

hACE2 Antibody Blocking Assay.

15,000 Vero E6 cells per well were seeded in a white opaque 96-well plate (Corning). On the next day, cells were wash three times with PBS to remove any residual FBS and followed by 1-hour treatment with goat anti-human ACE2 antibody (R&D Systems) or anti-hDDP4 antibody (R&D Systems) (both antibodies were prepared in OptiMEM medium to the given concentrations). Afterwards, cells were infected with SARS-CoV-2-Nluc (MOI 0.5). At 6 h post-infection, cells were washes twice and followed by the addition of 50 μl Nano luciferase substrate (Promega). After 5 minutes of incubation at room temperature, luciferase signals were measured using a Synergy™ Neo2 microplate reader (BioTek) per the manufacturer's instructions.

Immunofluorescence Assay.

Cells were seeded on a 4-well chamber slide. At 24 h post-seeding, cells were fixed and permeabilized with 0.1% Triton X-100. After 1 h-blocking with PBS+1% FBS, cellular hACE2 was probed firstly by goat anti-human ACE2 antibody (R&D Systems). After three times of PBS washes, the cells were incubated with donkey anti-goat IgG conjugated with Alexa Fluor® 488 (ThermoFisher Scientific). Finally, the fluorescence images were acquired using the Nikon Ti2-E inverted microscope armed with a 60× objective.

SARS-CoV-2-Nluc Neutralization Assay.

Vero E6 cells (15,000 per well in medium containing 2% FBS) were plated into a white opaque 96-well plate (Corning). At 16 h post-seeding, 30 μl of 2-fold serial diluted human sera were mixed with 30 μl of SARS-CoV-2-Nluc (MOI 0.5) and incubated at 37° C. for 1 hour. Afterwards, 50 μl of virus-sera complexes were transferred to each well of the 96-well plate. After 4 h of incubation at 37° C., cells were washed twice followed by the addition of Nano luciferase substrate (Promega). Luciferase signals were measured using a Synergy™ Neo2 microplate reader (BioTek) per the manufacturer's instructions. The relative luciferase signal was calculated by normalizing the luciferase signals of serum-treated groups to those of the no-serum controls. The concentration that reduces the 50% luciferase signals (NT₅₀) were estimated using a four-parameter logistic regression model from the Prism 8 software (GraphPad Software Inc., San Diego CA).

Plaque Reduction Neutralization Test (PRNT).

Approximately 1.2×10⁶ Vero E6 cells were seeded to each well of 6-well plates. On the following day, 100 PFU of infectious clone-derived wild-type SARS-CoV-2 was incubated with serially diluted serum (total volume of 200 μl) at 37° C. for 1 h. The virus-serum mixtures were transferred to the pre-seeded Vero E6 cells in 6-well plates. After incubation at 37° C. for 1 h, 2 ml of 2% high gelling temperature agar (SeaKem) in DMEM with 5% FBS and 1% P/S was added to the infected cells per well. After 2-day incubation, 2 ml of neutral red (1 g/l in PBS; Sigma) was added to the agar-covered cells. After another 5-h incubation, neutral red was removed, and individual plaques were counted for NT₅₀ calculation. Each specimen was tested in duplicates.

SARS-CoV-2-Nluc Antiviral Assay.

Vero E6 or A549-hACE2 cells (12,000 cells per well in phenol-red free medium containing 2% FBS) were plated into a white opaque 96-well plate (Corning). On the next day, 2-fold serial dilutions of compounds were prepared in DMSO. The compounds were further diluted as 100 folds in the phenol-red free culture medium containing 2% FBS. Cell culture fluids were removed and incubated with 50 μl of diluted compound solutions and 50 μl of SARS-CoV2-Nano viruses (MOI 0.025). At 48 h post-infection, 50 μl Nano luciferase substrates (Promega) were added to each well. Luciferase signals were measured using a Synergy™ Neo2 microplate reader. The relative luciferase signals were calculated by normalizing the luciferase signals of the compound-treated groups to that of the DMSO-treated groups (set as 100%). The relative luciferase signal (Y axis) versus the log 10 values of compound concentration (X axis) was plotted in software Prism 8. The EC₅₀ (compound concentration for reducing 50% of luciferase signals) were calculated using a nonlinear regression model (four parameters). Experiments were performed in duplicates. The entire screening assay for each compound was repeated once.

Cytotoxicity Assay.

Vero or A549-hACE2 cells (5,000 cells per well in phenol-red free medium containing 2% FBS) were plated into a clear flat bottom 96-well plate (Nunc). On the next day, 2-fold serial dilutions of compounds were prepared in DMSO. The compounds were further diluted as 100 folds. 50 μl diluted compound solutions were added to each well of the cell plates. At 72 h post-treatment, 4 μl of Cell Counting Kit-8 (CCK-8; Sigma-Aldrich) was added to each well. After incubation at 37° C. for 90 min, absorbance at 450 nm was measured using the Cytation5 multi-mode microplate reader (BioTek). The relative cell viability was calculated by normalizing the absorbance of the compound-treated groups to that of the DMSO-treated groups (set as 100%). The relative cell viability (Y axis) versus the log 10 values of compound concentration (X axis) were plotted in software Prism 8. The CC₅₀ (compound concentration for reducing 50% of cell viability) were calculated using a nonlinear regression model (four parameters). Experiments were performed in duplicates. The cytotoxicity assay for each compound was repeated once.

### Example 3

### A High-Throughput Neutralizing Antibody Assay for COVID-9 Diagnosis and Vaccine Evaluation

Virus neutralization remains the gold standard for determining antibody efficacy. Therefore, a high-throughput assay to measure SARS-CoV-2 neutralizing antibodies is urgently needed for COVID-19 serodiagnosis, convalescent plasma therapy, and vaccine development. Here we report on a fluorescence-based SARS-CoV-2 neutralization assay that detects SARS-CoV-2 neutralizing antibodies in COVID-19 patient specimens and yields comparable results to plaque reduction neutralizing assay, the gold standard of serological testing. The fluorescence-based neutralization assay is specific to measure COVID-19 neutralizing antibodies without cross reacting with patient specimens with other viral, bacterial, or parasitic infections. Collectively, our approach offers a rapid platform that can be scaled to screen people for antibody protection from COVID-19, a key parameter necessary to safely reopen local communities.

Results

A High-Throughput Fluorescence-Based Neutralization Assay.

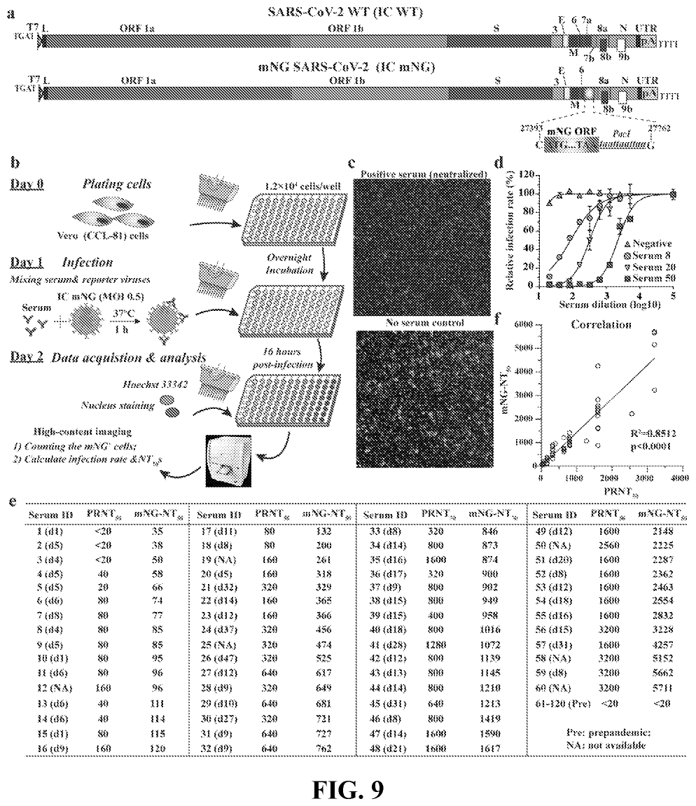

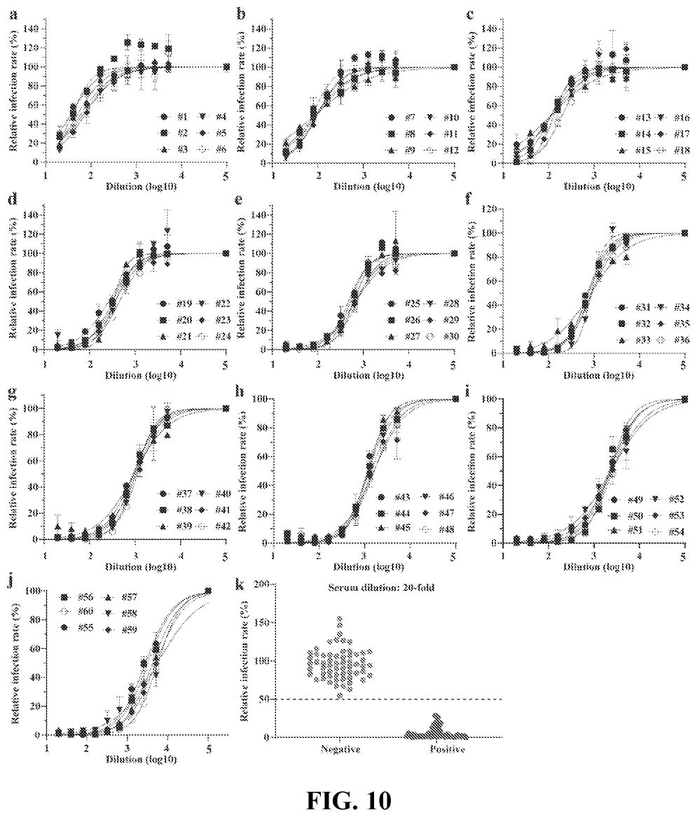

To fill in the gap for COVID-19 serodiagnosis and vaccine evaluation, we developed a fluorescence-based assay that rapidly and reliably measures neutralization of a reporter SARS-CoV-2 by antibodies from patient specimens. The assay was built on a stable mNeonGreen (mNG) SARS-CoV-2 where the mNG gene was engineered at the ORF7 of the viral genome (FIG. 9A) (Xie et al. Cell Host Microbe, 2020). FIG. 9B depicts the flowchart of the reporter neutralization assay in a 96-well format. Briefly, patient sera were serially diluted and incubated with the reporter virus. After incubation at 37° C. for 1 h, Vero CC-81 cells (pre-seeded in a 96-well plate) were infected with the virus/serum mixtures at a multiplicity of infection (MOI) of 0.5. At 16 h post-infection, the mNG-positive cells were quantitated using a high-content imaging reader (FIG. 9B). It should be noted that Vero CC-81 cells, not Vero E6 cells, were chosen for the mNG assay to enable accurate quantification of fluorescent cells. Sixty COVID-19 serum specimens from RT-PCR-confirmed patients and sixty non-COVID-19 serum samples (archived before COVID-19 emergence) were analyzed using the reporter virus. For some COVID-19-positive specimens, the sample collection days post viral RT-PCR positive were available and are indicated in Table 5. After reporter viral infection, the cells turned green in the absence of serum (FIG. 9C, bottom panel); in contrast, incubation of the reporter virus with COVID-19 patient serum decreased the number of fluorescent cells (top panel). A dose response curve was obtained between the number of fluorescent cells and the fold of serum dilution (FIG. 9D and FIG. 10), which allowed for determination of the dilution fold that neutralized 50% of fluorescent cells (NT₅₀). The reporter assay rapidly diagnosed one hundred and twenty specimens in less than 20 h: all sixty COVID-19 sera (specimens 1-60) showed positive NT₅₀ of 35 to 5711, and all sixty non-COVID-19 sera (specimens 61-120) showed negative NT₅₀ of <20 (Table 5).

TABLE 5
Comparison of neutralization titers of patient sera analyzed
by reporter assay and plaque reduction assay*¶

| Serum-ID¤ | PRNT [symbol] ¤ | mNG-NT [symbol] ¤ | Serum-ID¤ | PRNT [symbol] ¤ | mNG-NT [symbol] ¤ | Serum-ID¤ | PRNT [symbol] ¤ |
|---|---|---|---|---|---|---|---|
| 1-(d1)¤ | <20¤ | 35¤ | 17-(d11)¤ | 80¤ | 132¤ | 33-(d8)¤ | 320¤ |
| 2-(d5)¤ | <20¤ | 38¤ | 18-(d8)¤ | 80¤ | 200¤ | 34-(d14)¤ | 800¤ |
| 3-(d4)¤ | <20¤ | 50¤ | 19-(NA)¤ | 160¤ | 261¤ | 35-(d16)¤ | 1600¤ |
| 4-(d5)¤ | 40¤ | 58¤ | 20-(d5)¤ | 160¤ | 318¤ | 36-(d17)¤ | 320¤ |
| 5-(d5)¤ | 20¤ | 66¤ | 21-(d32)¤ | 320¤ | 329¤ | 37-(d9)¤ | 800¤ |
| 6-(d6)¤ | 80¤ | 74¤ | 22-(d14)¤ | 160¤ | 365¤ | 38-(d15)¤ | 800¤ |
| 7-(d8)¤ | 80¤ | 77¤ | 23-(d12)¤ | 160¤ | 366¤ | 39-(d15)¤ | 400¤ |
| 8-(d4)¤ | 80¤ | 85¤ | 24-(d37)¤ | 320¤ | 456¤ | 40-(d18)¤ | 800¤ |
| 9-(d5)¤ | 80¤ | 85¤ | 25-(NA)¤ | 320¤ | 474¤ | 41-(d28)¤ | 1280¤ |
| 10-(d1)¤ | 80¤ | 95¤ | 26-(d47)¤ | 320¤ | 525¤ | 42-(d12)¤ | 800¤ |
| 11-(d5)¤ | 80¤ | 96¤ | 27-(d12)¤ | 640¤ | 617¤ | 43-(d13)¤ | 800¤ |
| 12-(NA)¤ | 160¤ | 96¤ | 28-(d9)¤ | 320¤ | 649¤ | 44-(d14)¤ | 800¤ |
| 13-(d6)¤ | 40¤ | 111¤ | 29-(d10)¤ | 640¤ | 681¤ | 45-(d31)¤ | 640¤ |
| 14-(d [symbol] )¤ | 40¤ | 114¤ | 30-(d27)¤ | 320¤ | 721¤ | 46-(d8)¤ | 800¤ |
| 15-(d1)¤ | 80¤ | 115¤ | 3 [symbol] -(d9)¤ | 640¤ | 727¤ | 47-(d14)¤ | 1600¤ |
| 16-(d9)¤ | 160¤ | 120¤ | 32-(d9)¤ | 640¤ | 762¤ | 48-(d21)¤ | 1600¤ |

| mNG-NT [symbol] ¤ | Serum-ID¤ | PRNT [symbol] ¤ | mNG-NT [symbol] ¤ | Serum-ID¤ | PRNT [symbol] ¤ | mNG-NT [symbol] ¤ |
|---|---|---|---|---|---|---|
| 846¤ | 33-(d8)¤ | 320¤ | 846¤ | 49-(d12)¤ | 1600¤ | 2148¤ |
| 873¤ | 34-(d14)¤ | 800¤ | 873¤ | 50-(NA)¤ | 2560¤ | 2225¤ |
| 874¤ | 35-(d16)¤ | 1600¤ | 874¤ | 51-(d20)¤ | 1600¤ | 22 [symbol] 7¤ |
| 900¤ | 36-(d17)¤ | 320¤ | 900¤ | 52-(d8)¤ | 1600¤ | 2362¤ |
| 902¤ | 37-(d9)¤ | 800¤ | 902¤ | 53-(d12)¤ | 1600¤ | 2463¤ |
| 949¤ | 38-(d15)¤ | 800¤ | 949¤ | 54-(d18)¤ | 1600¤ | 2554¤ |
| 958¤ | 39-(d15)¤ | 400¤ | 958¤ | 55-(d16)¤ | 1600¤ | 2 [symbol] 32¤ |
| 1016¤ | 40-(d18)¤ | 800¤ | 1016¤ | 56-(d15)¤ | 3200¤ | 3228¤ |
| 1072¤ | 41-(d28)¤ | 1280¤ | 1072¤ | 57-(d31)¤ | 1600¤ | 4257¤ |
| 1139¤ | 42-(d12)¤ | 800¤ | 1139¤ | 58-(NA)¤ | 3200¤ | 5152¤ |
| 1145¤ | 43-(d13)¤ | 800¤ | 1145¤ | 59-(d8)¤ | 3200¤ | 5 [symbol] 62¤ |
| 1210¤ | 44-(d14)¤ | 800¤ | 1210¤ | 60-(NA)¤ | 3200¤ | 5711¤ |
| 1213¤ | 45-(d31)¤ | 640¤ | 1213¤ | 61-120-(Pre)¤ | <20¤ | <20¤ |
| 1419¤ | 46-(d [symbol] )¤ | 800¤ | 1419¤ | ¤ | ¤ | ¤ |
| 1590¤ | 47-(d14)¤ | 1600¤ | 1590¤ | ¤ | ¤ | ¤ |
| 1617¤ | 48-(d21)¤ | 1600¤ | 1617¤ | ¤ | ¤ | ¤ |

*A total of 120 patient sera were analyzed, including 60 specimens from RT-PCR-confirmed patients (specimens 1-60) and 60 negative specimens (specimens 61-120) that were collected before COVID-19-pandemic (prepandemic).
¤The NT₅₀ and PRINT₅₀ values were derived from the reporter virus assay and conventional PRNT assay, respectively.
¶Sample collection days post-after RT-PCR positive test are indicated in parentheses. For some COVID-19-positive specimens, the sample collection days post after RT-PCR positive test are not available (NA). [symbol]
indicates data missing or illegible when filed

Assay Validation by Plaque Reduction Test.

To validate the reporter virus neutralization results, the conventional PRNT was performed on the same set of patient specimens. All sixty negative sera (specimens 61-120) exhibited PRNT₅₀ of <20 (Table 5). Among the sixty positive specimens, fifty-seven sera (specimens 4-60) showed PRNT₅₀ of 40 to 3200, whereas three sera (specimens 1-3) exhibited PRNT₅₀ of <20 (Table 5). The discrepancy between the PRNT₅₀ and NT₅₀ values for specimens 1-3 is likely due to the early infection time (within 5 days post RT-PCR positive) when neutralizing antibodies just began to develop; this discrepancy suggests that the mNG SARS-CoV-2 assay has a higher sensitivity than the conventional PRNT assay. Nevertheless, a strong correlation was observed between the reporter virus and PRNT results, with a correlation efficiency R² of 0.85 (FIG. 9E). The results demonstrate that when diagnosing patient specimens, the reporter virus assay delivers neutralization results comparable to the PRNT assay, the gold standard of serological testing.

Assay Specificity.

The specificity of reporter neutralization assay was evaluated using potentially cross-reactive sera and interfering substances (Table 6). Two groups of specimens were tested for cross reactivity. Group I included 150 clinical sera from patients with antigens or antibodies against different viruses, bacteria, and parasites. These human specimens were obtained according to two types of diagnostic results: Some samples were tested positive for antibodies against specific pathogens (e.g., anti-Chikungunya virus; this group of samples are indicated by prefix “anti” in Table 6); other specimens were collected within one to six months after the patients were tested positive on pathogen antigens or nucleic acids (e.g., Cryptococcus neoformans antigen; this group of samples are not indicated by prefix in Table 6). Group II consisted of 19 samples with albumin, elevated bilirubin, cholesterol, rheumatoid factor, and autoimmune nuclear antibodies. None of these specimens cross neutralized mNG SARS-CoV-2 (Table 6), including the four common cold coronaviruses (NL63, 229E, OC43, and HUK1). Despite the low number of common cold coronavirus serum specimens, our result is consistent with the recent reports that sera from common cold coronavirus patients did not cross react with SARS-CoV-2 (Amanat et al. Nat Med, 2020; Khan et al. bioRxiv 2020). More specimens are required to further validate the cross reactivity, particularly between SARS-CoV-2 and other human coronaviruses, including SARS-CoV-1 and MERS-CoV.

TABLE 6
Cross reactivity of mNG SARS-COV-2 neutralization assay

| | | Number of |
|---|---|---|
| | Sample | mNG tested |
| *Immune sera and ^{#}interfering substances | number | positive |
| Adenovirus | 1 | 0 |
| Anti-Chikungunya virus | 4 | 0 |
| Cryptococcus neoformans antigen | 2 | 0 |
| Anti-Cytomegalovirus | 8 | 0 |
| Anti-Dengue virus | 5 | 0 |
| Anti-Epstein Barr Virus: capsid or nuclear antigen | 8 | 0 |
| Anti-Hepatitis A virus | 5 | 0 |
| Anti-Hepatitis B virus: surface antigen | 15 | 0 |
| Anti-Hepatitis C virus | 3 | 0 |
| Anti-Herpes simplex virus 1 | 7 | 0 |
| Anti-Herpes simplex virus 2 | 5 | 0 |
| Human coronavirus 229E | 1 | 0 |
| Human coronavirus HKU1 | 5 | 0 |
| Human coronavirus NL63 | 1 | 0 |
| Human coronavirus OC43 | 4 | 0 |
| Anti-Human immunodeficiency virus 1 | 10 | 0 |
| Human rhinovirus | 3 | 0 |
| Influenza B virus | 2 | 0 |
| Anti-Measles virus | 7 | 0 |
| Anti-Mumps virus | 5 | 0 |
| Parainfluenza virus 2 | 1 | 0 |
| Parainfluenza virus 4 | 1 | 0 |
| Anti-Parvovirus B19 | 4 | 0 |
| Respiratory syncitial virus | 1 | 0 |
| Anti-Rubella virus | 12 | 0 |
| Anti-Syphilis | 5 | 0 |
| Anti-Toxoplasma | 2 | 0 |
| Anti-Typhus Fever | 1 | 0 |
| Anti-Varicella zoster virus | 13 | 0 |
| Anti-West Nile Virus | 3 | 0 |
| Anti-Yellow fever virus: vaccination | 2 | 0 |
| Anti-Zika virus | 4 | 0 |
| ^{#}Albumin (4.5 g/dL) | 3 | 0 |
| ^{#}Elevated bilirubin conjugated (>0.4 mg/dL) | 3 | 0 |
| ^{#}Elevated bilirubin unconjugated (>0.8 mg/dL) | 3 | 0 |
| ^{#}Elevated cholesterol (>200 mg/dL) | 3 | 0 |
| ^{#}Elevated rheumatoid factor (>100 IU/mL) | 3 | 0 |
| ^{#}Anti-Nuclear antibodies | 4 | 0 |

*A total of 150 sera with antigens or antibodies against different infections (or immunizations) were tested against mNG SARS-COV-2 neutralization assay. The immune sera are listed in alphabetical order. Samples tested positive for antibodies against specific pathogens are indicated with prefix “anti“, whereas samples tested positive on antigens or pathogen nucleic acids are not indicated with prefix. For the latter group, the specimens were collected within one to six months after the antigen or PCR tested positive.
^{#}A total of 19 samples tested for interfering substances and autoimmune disease nuclear antibodies.

### Methods

Cells.

Vero (ATCC ° CCL-81) and Vero E6 (ATCC® CRL-1586) were purchased from the American Type Culture Collection (ATCC, Bethesda, MD), and maintained in a high-glucose Dulbecco's modified Eagle's medium (DMEM) supplemented with 10% fetal bovine serum (FBS; HyClone Laboratories, South Logan, UT) and 1% penicillin/streptomycin at 37° C. with 5% CO₂. All culture medium and antibiotics were purchased from ThermoFisher Scientific (Waltham, MA). All cell lines were tested negative for mycoplasma.

mNG SARS-CoV-2.

The virus stock of mNG SARS-CoV-2 was produced using an infectious cDNA clone of SARS-CoV-2 in which the ORF7 of the viral genome was replaced with reporter mNG gene (Xie et al. Cell Host Microbe, 2020). After rescued from the genome-length viral RNA-electroporated cells, the viral stock was prepared by amplifying the mNG SARS-CoV-2 on Vero E6 cells for one or two rounds. The titer of the virus stock was determined by a standard plaque assay.

Human Sera and Interfering Substances.

The research protocol regarding the use of human serum specimens was reviewed and approved by the University of Texas Medical Branch (UTMB) Institutional Review Board. The approved IRB protocol number is 20-0070. All human serum specimens were obtained at the UTMB. All specimens were de-identified from patient information. A total of forty de-identified convalescent sera from COVID-19 patients (confirmed with viral RT-PCR positive) were tested in this study. Ten non-COVID-19 sera, collected before COVID-19 emergence (Shan et al. J Clin Microbiol 55, 3028-3036, 2017; Shan et al. EBioMedicine 17, 157-162, 2017), were also tested in the reporter virus and PRNT assays. For testing cross reactivity, a total of 138 de-identified specimens from patients with antigens or antibodies against different viruses, bacteria, and parasites were tested in the mNG SARS-COV-2 neutralization assay (Table 6). For testing interfering substances, nineteen de-identified serum specimens with albumin, elevated bilirubin, cholesterol, rheumatoid factor, and autoimmune nuclear antibodies were tested in the reporter neutralization assay. All human sera were heat-inactivated at 56° C. for 30 min before testing.

mNG SARS-CoV-2 Reporter Neutralization Assay.

Vero CCL-81 cells (1.2×10⁴) in 50 μl of DMEM (Gibco) containing 2% FBS (Hyclone) and 100 U/ml Penicillium-Streptomycin (P/S; Gibco) were seeded in each well of black μCLEAR flat-bottom 96-well plate (Greiner Bio-One™). Vero CC-81 cells, not Vero E6 cells, were selected for the mNG SARS-COV-2 assay to facilitate accurate quantification of fluorescent cells by high-content imaging. The cells were incubated overnight at 37° C. with 5% CO₂. On the following day, each serum was 2-fold serially diluted in 2% FBS and 100 U/ml P/S DMEM, and incubated with mNG SARS-CoV-2 at 37° C. for 1 h. The virus-serum mixture was transferred to the Vero E6 cell plate with the final multiplicity of infection (MOI) of 0.5. For each serum, the starting dilution was 1/20 with nine 2-fold dilutions to the final dilution of 1/5120. After incubating the infected cells at 37° C. for 16 h, 25 μl of Hoechst 33342 Solution (400-fold diluted in Hank's Balanced Salt Solution; Gibco) were added to each well to stain cell nucleus. The plate was sealed with Breath-Easy sealing membrane (Diversified Biotech), incubated at 37° C. for 20 min, and quantified for mNG fluorescence on Cytation™ 7 (BioTek). The raw images (2×2 montage) were acquired using 4× objective, processed, and stitched using the default setting. The total cells (indicated by nucleus staining) and mNG-positive cells were quantified for each well. Infection rates were determined by dividing the mNG-positive cell number to total cell number. Relative infection rates were obtained by normalizing the infection rates of serum-treated groups to those of non-serum-treated controls. The curves of the relative infection rates versus the serum dilutions (log 10 values) were plotted using Prism 8 (GraphPad). A nonlinear regression method was used to determine the dilution fold that neutralized 50% of mNG fluorescence (NT₅₀). Each serum was tested in duplicates. All mNG SARS-CoV-2 reporter neutralization assay was performed at the BSL-3 facility at UTMB.

Plaque Reduction Neutralization Test (PRNT).

Vero E6 cells (1.2×10⁶ per well) were seeded to 6-well plates. On the following day, 100 PFU of infectious clone-derived wild-type SARS-CoV-2 was incubated with serially diluted serum (total volume of 200 μl) at 37° C. for 1 h. The virus-serum mixture was added to the pre-seeded Vero E6 cells. After 1 h 37° C. incubation, 2 ml of 2% high gel temperature agar (SeaKem) in DMEM containing 5% FBS and 1% P/S was added to the infected cells. After 2 days of incubation, 2 ml neutral red (1 g/l in PBS; Sigma) was added to the agar-covered cells. After another 5-h incubation, neutral red was removed. Plaques were counted for NT₅₀ calculation. Each serum was tested in duplicates. The PRNT assay was performed at the BSL-3 facility at UTMB.

Statistical Analysis.

The correlation of the NT₅₀ values from mNG reporter SARS-CoV-2 assay and the PRNT₅₀ values from plaque neutralization assay was analyzed using a linear regression model in the software Prism 8 (GraphPad). Pearson correlation coefficient and two-tailed p value are calculated using the default settings in the software Prism 8.

### Example 4

### Standard Operation Procedure (SOP) for mNeonGreen SARS-CoV-2 Neutralization Assay

Reagents and Equipment
Greiner Bio-One™ CellStar™ 96-Well, Cell Culture-Treated, Flat-Bottom (Greiner Bio-One™ 655090)
Corning™ Clear Polystyrene 96-Well Microplates, round bottom (Corning Cat. No: 3799)
Culture medium: DMEM (Gibco Cat. No: 11965) supplemented with 10% FBS, 1% p/S
Assay medium: phenol red-free DMEM (Gibco Cat. No: 31053028), supplemented with 2% GlutaMAX (Gibco, Cat. No: 35050079), 2% FBS, 1% P/S.
Fetal Bovine Serum (Hyclone Cat. No.: SH30071)
Penicillium-Streptomycin (P/S) (10,000 U/ml) (Gibco Cat. No: 15140122)
Gibco™ Trysin-EDTA (0.25%), phenol red (Gibco Cat. No.: 25200072)
Phosphate Buffered Saline (PBS) solution, pH 7.4 (Gibco Cat. No.: 10010049)
Hoechst 33342 Solution (ThermoFisher Scientific, Cat No: 62249)
Reagent Reservoirs 25 ml (Gilson Cat. No.: F267660) & 50 ml (Gilson Cat. No.: F267670)
Cell Counting slides for TC10™/TC20™ Cell Counter, dual-chamber (Bio-Rad Cat. No.: 1450011)
Corning™ Cell Culture Treated flasks (Corning™ Cat. No.: 431080)
VACUBOY Hand Operator (INTEGRA Biosciences)
Eppendorf Xplorer pipette, electronic 12 channel pipette 15-300 μL (Eppendorf Cat. No.: 4861000031)
TC20™ Automated Cell Counter (Bio-Rad)
Tissue culture CO₂ incubator
Cytation™ 7 Cell Imaging Multi-Mode Reader (BioTek)

Prepare Cells Prior to Infection (on Day 0)

Seed cells into 96-well plate Solid Black Polystyrene Microplates with clear bottom.

Cells are grown a T-175 flask. Upon seeing cells, remove the medium from the cells using the VACUBOY.

Briefly rinse the cell layer with 25 ml PBS to remove all traces of serum. Remove PBS using the VACUBOY. Repeat the PBS wash once. Note: make sure rinse every corner of the flask to get rid of any trypsin inhibitor.

Add 3 ml Trypsin-EDTA solution. Observe cells under an inverted microscope until cell layer is dispersed (usually within 2 minutes at room temperature).

Tap the flask vigorously to detach cells, add 12 ml of complete growth medium and pipet up and down gently to disperse the cell suspension. Transfer the cell suspension into a 50-ml falcon tube.

Centrifuge at room temperature for 3 min at 1,200×rpm (300 g).

Remove media completely.

Resuspend cells in 10 ml assay medium. Disperse cells by pipetting up and down.

Count the cell number using the cell counter (C-Chip DHC-N01-5). Count live cells by mixing 50 μl of trypan blue with 50 μl of cell suspension.

Dilute cells to a final concentration of 2.4×10⁵ cells/ml. Add 50 μl of the diluted cell suspension to each well of a 96-well plate to reach 1.2×10⁴ cells/well.

Incubate the plates at 37° C. with 5% CO₂.

Prepare Serum Dilutions and Infection (on Day 1)

Heat inactivate all the sera at 56° C. for 30 min.

Prepare serial dilutions of serum in a round bottom 96-well plate. Prepare 10 serials of 2-fold dilutions. The highest dilution is 10-folds. Prepare dilutions in duplicates. See the diagram below.

| D1 | D2 | D3 | D4 | D5 | D6 | D7 | D8 | D9 | D10 |
|---|---|---|---|---|---|---|---|---|---|
| 10 | 20 | 40 | 80 | 160 | 320 | 640 | 1280 | 2156 | 0 |

D1: 6 μl serum + 54 μl of assay medium;
D2-D9: 30 μl diluted samples + 30 μl assay medium.
D10: 30 μl assay medium

Transfer 30 μl from column #D1 to column #D2 using the electronic pipets with settings of P/M, 30/50 and speed 6/4, 3×. Repeat the step for columns D3 to D9.

At dilution D9, take 30 μl from after dilution and discard. The total volume of the dilute serum should be 30 μl/well.

Sera dilution plate set-up. Samples are run in duplicates.

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | | | | | | | | | |
|---|---|---|---|---|---|---|---|---|---|---|---|
| | A | | | | | | | | | | |
| Sample | B | (+) | 1:20 | 1:40 | 1:80 | 1:160 | 1:320 | 1:640 | 1:1280 | 1:2560 | (−)cells |
| 1 | | UN | | | | | | | | | |
| Sample | C | (+) | 1:20 | 1:40 | 1:80 | 1:160 | 1:320 | 1:640 | 1:1280 | 1:2560 | (−)cells |
| 1 | | UN | | | | | | | | | |
| Sample | D | (+) | 1:20 | 1:40 | 1:80 | 1:160 | 1:320 | 1:640 | 1:1280 | 1:2560 | (−)cells |
| 2 | | UN | | | | | | | | | |
| Sample | E | (+) | 1:20 | 1:40 | 1:80 | 1:160 | 1:320 | 1:640 | 1:1280 | 1:2560 | (−)cells |
| 2 | | UN | | | | | | | | | |
| Sample | F | (+) | 1:20 | 1:40 | 1:80 | 1:160 | 1:320 | 1:640 | 1:1280 | 1:2560 | (−)cells |
| 3 | | UN | | | | | | | | | |
| Sample | G | (+) | 1:20 | 1:40 | 1:80 | 1:160 | 1:320 | 1:640 | 1:1280 | 1:2560 | (−)cells |
| 3 | | UN | | | | | | | | | |
| | H | | | | | | | | | | |

In BSL-3, add 30 μl of diluted SARS-CoV2-mNG virus (MOI is 0.5) to each well of the serum. Mix the serum with virus solutions thoroughly by gentle pipetting.

Incubate the plates at 37° C. for 1 h.

Transfer 50 μl virus-serum complexes to the 96-well plates seeded on day −1 (cell plates containing 50 μl of culture media per well, after adding the diluted reporter virus the total volume of each well will be 100 μl). Mix using an electronic 12-channel pipette (setting, P/M, 50/75, speed 3/3, 2×).

### Example: Cells+Ab+Reporter Virus Plate Setup (Black Plate)

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | | | | | | | | | | |
|---|---|---|---|---|---|---|---|---|---|---|---|---|
| | A | | | | | | | | | | | |
| Sample 1 | B | +V | +V | +V | +V | +V | +V | +V | +V | +V | +V | No virus |
| Sample 1 | C | +V | +V | +V | +V | +V | +V | +V | +V | +V | +V | No virus |
| Sample 2 | D | +V | +V | +V | +V | +V | +V | +V | +V | +V | +V | No virus |
| Sample 2 | E | +V | +V | +V | +V | +V | +V | +V | +V | +V | +V | No virus |
| Sample 3 | F | +V | +V | +V | +V | +V | +V | +V | +V | +V | +V | No virus |
| Sample 3 | G | +V | +V | +V | +V | +V | +V | +V | +V | +V | +V | No virus |
| | H | | | | | | | | | | | |

Incubate at 37° C. for 20 h.

Data Acquisition and Analysis (on Day 2)

At 20 h post-infection, in BSL-3 facility, add 25 μl diluted Hoechst 33342 Solution (diluted at 400× in PBS) to each well of the 96-well plate.

After incubating at 15 min at 37° C., acquire the images with both DAPI staining (in blue) and mNG signals (green) using Cytation™ 7 Cell Imaging Multi-Mode Reader (BioTek) according to the manufacturer's instructions.

Count the total cell numbers: mean intensities (in blue) within the primary mask ≥5000.

Count the mNG-positive cells: mean intensities (in green) within the secondary mask ≥3100. The threshold of green intensity was selected to distinguish mNG-positive signals from the background.

Determine the infection rate: (100×mNG-positive cell number/total cell number) %. Optimization may be required to achieve the infection rate in the no-serum controls at 10%-30% for robustness. The cell controls (without viruses) should be <1%.

Plot the neutralization values in Prism to calculate the NT₅₀s and Hislopes.

Normalize the infection rate to the no-serum control wells.

Plot the relative infection rate versus the dilution (log 10 values) in the Prism software 8.3.

Fit the curve and calculate the Neutralizing titers (NT₅₀) using the nonlinear regression model: log(inhibitor) vs. response-variable slope (four parameters) with constrain of bottom to 0 and top to 100.

Data interpretation. NT₅₀<20: negative; NT₅₀≥20: Positive

1. A recombinant DNA expression cassette comprising a SARS-CoV-2 nucleic acid segment that is at least 95% identical to the nucleic acid sequence of SEQ ID NO:1, SEQ ID NO:2, or SEQ ID NO:3.

2. The expression cassette of claim 1 wherein the SARS-CoV-2 nucleic acid segment that is at least 99.99% identical to the nucleic acid sequence of SEQ ID NO:1, SEQ ID NO:2, or SEQ ID NO:3.

3. The expression cassette of claim 1, wherein the SARS-CoV-2 nucleic acid segment identical to the nucleic acid sequence of SEQ ID NO:1, SEQ ID NO:2, or SEQ ID NO:3.

4. The expression cassette of claim 1, wherein the expression cassette is comprised in a plasmid backbone.

5. The expression cassette of claim 1, wherein the SARS-CoV-2 nucleic acid segment is operatively coupled to a heterologous promoter segment.

6. A host cell comprising an expression cassette of claim 1.

7. A recombinant SARS-CoV-2 genome comprising an expression cassette of claim 3.

8. The recombinant SARS-CoV-2 of claim 7, wherein the heterologous nucleic acid segment encodes a reporter protein.

9. The recombinant SARS-CoV-2 of claim 8, wherein the reporter protein is a fluorescent or luminescent protein.

10. The recombinant SARS-CoV-2 of claim 9, wherein the fluorescent protein is mNeonGreen protein.

11. The recombinant SARS-CoV-2 of claim 9, wherein the luminescent protein is nanoluciferase protein.

12. A recombinant cDNA comprising a nucleotide sequence that is at least 95% identical to SEQ ID NO:1, SEQ ID NO:2, or SEQ ID NO:3.

13. An assay for SARS-CoV-2 replication comprising:
contacting a cultured cell expressing or containing a nucleotide sequence at least 95% identical to SEQ ID NO:3 forming a test cell;
contacting the test cell with a agent; and
assessing the replication of SARS-CoV-2 in the presence of the test agent.

14. The assay of claim 13, wherein the rSARS-COV-2 is a mNeonGreen (mNG) reporter SARS-COV-2.

15. The assay of claim 13, wherein the cultured cell is a Vero cell.

16. The assay of claim 13, wherein the cultured cell is assayed in a multi-well plate.

17. The assay of claim 16, wherein the multi-well plate is a 96 well microtiter plate.

18. The assay of claim 13, wherein the infected cells are incubated for about 12, 24, 36, or 48 hours before measuring the reporter signal.

19. A recombinant SARS-CoV-2 comprising a SARS-CoV-2 genome of claim 7.


SEQUENCE LISTING (9 sequences)

SEQ ID NO: 1 - DNA, 29903 bp, Coronavirus
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - DNA, 29899 bp, Coronavirus
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 3 - DNA, 30250 bp, Coronavirus
  misc_feature (27394)..(28112): reporter gene inserted in place of segment 27394 to 27761
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatggtgagcaaaggagaagaggacaacatggcatcactcccagctacacatgagctgcatatcttcggatccatcaacggagtggacttcgatatggtgggacagggtacagggaacccaaacgacggatacgaggagttgaacctgaagagtaccaagggagacctgcagttctcaccatggatactcgtcccacatataggatacggctttcatcagtacctgccctatccagacggaatgtcacctttccaggcagccatggttgacgggagcggttaccaggtccacaggacaatgcagtttgaggacggagcctcattgaccgtgaactacagatatacctacgaaggaagccatatcaagggagaggctcaagtgaagggaactggattcccagcggacggacccgtgatgaccaacagtctgacggctgcagactggtgcagatccaaaaagacctacccaaatgacaagacaatcataagcaccttcaagtggtcatacactacaggaaacgggaagagatacaggagcactgccagaaccacatacactttcgccaagcctatggctgcaaactacctcaagaaccaacccatgtatgtgttcagaaagacagaactgaagcattctaagaccgaactgaacttcaaggagtggcagaaggcctttactgacgtgatgggaatggacgaactctacaagtaattaattaagaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 4 - DNA, 719 bp, Artificial Sequence
  recombinant polynucleotide
  CDS (1)..(717)
atggtgagcaaaggagaagaggacaacatggcatcactcccagctacacatgagctgcatatcttcggatccatcaacggagtggacttcgatatggtgggacagggtacagggaacccaaacgacggatacgaggagttgaacctgaagagtaccaagggagacctgcagttctcaccatggatactcgtcccacatataggatacggctttcatcagtacctgccctatccagacggaatgtcacctttccaggcagccatggttgacgggagcggttaccaggtccacaggacaatgcagtttgaggacggagcctcattgaccgtgaactacagatatacctacgaaggaagccatatcaagggagaggctcaagtgaagggaactggattcccagcggacggacccgtgatgaccaacagtctgacggctgcagactggtgcagatccaaaaagacctacccaaatgacaagacaatcataagcaccttcaagtggtcatacactacaggaaacgggaagagatacaggagcactgccagaaccacatacactttcgccaagcctatggctgcaaactacctcaagaaccaacccatgtatgtgttcagaaagacagaactgaagcattctaagaccgaactgaacttcaaggagtggcagaaggcctttactgacgtgatgggaatggacgaactctacaagtaattaattaa

SEQ ID NO: 5 - protein, 236 aa, Artificial Sequence
  Synthetic Construct
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYK

SEQ ID NO: 6 - DNA, 513 bp, Artificial Sequence
  recombinant polynucleotide
  CDS (1)..(513)
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcg

SEQ ID NO: 7 - protein, 171 aa, Artificial Sequence
  Synthetic Construct
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 8 - DNA, 1650 bp, Artificial Sequence
  recombinant polynucleotide
  CDS (1)..(1650)
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtataccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggtggtaagatcgccgtg

SEQ ID NO: 9 - protein, 550 aa, Artificial Sequence
  Synthetic Construct
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV